(19) United States
(12) Patent Application Publication
(10) Pub. No.: US 2007/0025997 A1
(43) Pub. Date: Feb. 1, 2007

(54) Use of biomolecular targets in the treatment and visualization of brain tumors

(76) Inventors: Usha Nagavarapu, San Jose, CA (US); David A. Shivak, San Francisco, CA (US); Daniel J. Chin, Foster City, CA (US); Erik D. Foehr, Novato, CA (US); Mirella Gonzalez-Zulueta, Pacifica, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/413,566

(22) Filed: Apr. 27, 2006

Related U.S. Application Data
(63) Continuation-in-part of application No. 10/407,365, filed on Apr. 3, 2003, now abandoned.
(60) Provisional application No. 60/369,743, filed on Apr. 3, 2002.
(60) Provisional application No. 60/676,344, filed on Apr. 28, 2005.

(51) Int. Cl. A61K 39/395; C12Q 1/68; G01N 33/574

(52) U.S. Cl. 424/155.1; 435/6; 435/7.23; 424/178.1

(57) ABSTRACT

The present invention relates to the use of proteins that are differentially expressed in primary brain tumor tissues, as compared to normal brain tissues, as biomolecular targets for brain tumor treatment therapies. Specifically, the present invention relates to the use of therapeutic and imaging agents, which specifically bind to one or more of the identified brain tumor protein targets. The present invention also provides compounds and pharmaceutically acceptable compositions for administration in the methods of the invention. Nucleic acid probes specific for the spliced mRNA encoding these variants and affinity reagents specific for the novel proteins are also provided.

### BACKGROUND OF THE INVENTION

Among tumors, those of the brain are considered to have one of the least favorable prognoses for long term survival: the average life expectancy of an individual diagnosed with a central nervous system (CNS) tumor is just eight to twelve months. Several unique characteristics of both the brain and its particular types of neoplastic cells create daunting challenges for the complete treatment and management of brain tumors. Among these are the physical characteristics of the intracranial space; the relative biological isolation of the brain from the rest of the body; the relatively essential and irreplaceable nature of the organ mass; and the unique nature of brain tumor cells.

The intracranial space and physical layout of the brain create significant obstacles to treatment and recovery. The brain is primarily comprised of astrocytes, which make up the majority of the brain mass, and serve as a scaffold and support for the neurons, which carry the actual electrical impulses of the nervous system, and a minor contingent of other cells, such as insulating oligodendrocytes that produce myelin. These cell types give rise to primary brain tumors, including astrocytomas, neuroblastomas, glioblastomas, oligodendrogliomas, and the like.

The brain is encased in the rigid shell of the skull, and is cushioned by the cerebrospinal fluid. Because of the relatively small volume of the skull cavity, minor changes in the volume of tissue in the brain can dramatically increase intracranial pressure, causing damage to the entire organ. Thus, even small tumors can have a profound and adverse affect on the brain's function. The cramped physical location of the cranium also makes surgery and treatment of the brain a difficult and delicate procedure. However, because of the dangers of increased intracranial pressure from the tumor, surgery is often the first strategy of attack in treating brain tumors.

In addition to its physical isolation, the brain is chemically and biologically isolated from the rest of the body by the “Blood-Brain-Barrier” (or BBB). This physiological phenomenon is due to the “tightness” of the epithelial cell junctions in the lining of the blood vessels in the brain. Nutrients, which are actively transported across the cell lining, can reach the brain, but other molecules from the bloodstream are excluded. This prevents toxins, viruses, and other potentially dangerous molecules from entering the brain cavity. However, it also prevents therapeutic molecules, including many chemotherapeutic agents that are useful in other types of tumors, from crossing into the brain. Thus, many therapies directed at the brain must be delivered directly into the brain cavity, e.g. by an Ommaya reservoir, or administered in elevated dosages to ensure the diffusion of an effective amount across the BBB.

With the difficulties of administering chemotherapies to the brain, radiotherapy approaches have also been attempted. However, the amount of radiation necessary to completely destroy potential tumor-producing cells also produce unacceptable losses of healthy brain tissue. The retention of patient cognitive function while eliminating the tumor mass is another challenge to brain tumor treatment. Neoplastic brain cells are often pervasive, and travel throughout the entire brain mass. Thus, it is impossible to define a true “tumor margin,” unlike, for example, in lung or bladder cancers. Unlike reproductive (ovarian, uterine, testicular, prostate, etc.), breast, kidney, or lung cancers, the entire organ, or even significant portions, cannot be removed to prevent the growth of new tumors. In addition, brain tumors are very heterogeneous, with different cell doubling times, treatment resistances, and other biochemical idiosyncrasies between the various cell populations that make up the tumor. This pervasive and variable nature greatly adds to the difficulty of treating brain tumors while preserving the health and function of normal brain tissue.

Although current surgical methods offer considerably better post-operative life for patients, current combination therapy methods (surgery, low-dosage radiation, and chemotherapy) have only improved the life expectancy of patients by one month, as compared to the methods of 30 years ago. Without effective agents to prevent the growth of brain tumor cells that are present outside the main tumor mass, the prognosis for these patients cannot be significantly improved. Although some immuno-affinity agents have been proposed and tested for the treatment of brain tumors, see, for example, the tenascin-targeting agents described in U.S. Pat. No. 5,624,659, these agents have not proven sufficient for the treatment of brain tumors. Thus, therapeutic agents which are directed towards new molecular targets, and are capable of specifically targeting and killing brain tumor cells, are urgently needed for the treatment of brain tumors.

Relevant Literature

Analysis of differential gene expression in glioblastoma may be found in, for example, Mariani et al. (2001) J Neurooncol 53(2):161-76; Markert et al. (2001) Physiol Genomics 5(1):21-33; Yano et al. (2000) Neurol Res 22(7):650-6; Kroes et al. (2000) Cancer Lett 156(2):191-8; and Reis et al. (2000) Am J Pathol 156(2):425-32, among others.

The receptor tyrosine kinase DDR1 (also referred to as MCK-10) is described in U.S. Pat. No. 6,051,397, Ullrich et al.

### SUMMARY OF THE INVENTION

The present invention provides methods and reagents for specifically targeting brain tumor neoplastic cells for both therapeutic and imaging purposes, by targeting the brain tumor target protein, DDR1, which is identified as being overexpressed in brain tumors, and thus allow for the selective inhibition of cell function or selective marking for visualization with therapeutic or visualizing compositions which have a specific affinity for these protein targets.

In one embodiment of the invention DDR1 expression is used as a specific marker for the diagnosis and treatment of grade II and/or grade III astrocytoma. Included in such methods is DDR1 of the DDR1a isotype, of the DDR1b isotype, of the DDR1d and DDR1e isotype, and soluble fragments of DDR1, e.g. cleaved at the RFRR protease recognition site.

Agents that bind to, or otherwise inhibit DDR1 function can inhibit the invasiveness of astrocytoma cells, and are effective in preventing the spread of brain tumors through extracellular matrix and basement membrane. Inhibitors can also target matrix metalloproteases that are induced by activation of DDR1, e.g. thiol, alkylcarbonyl, phosponamidate and hydroxamate MMP inhibitor compounds, such as marimastat and prinomastat.

In another embodiment of the invention, antibodies specific for DDR1 are used in therapy and/or diagnosis. The antibodies may be human or humanized antibodies, and can selectively bind an epitope present in a DDR1 specific sequence; the discoidin domain; the F5/8 type C domain; the RFRR protease recognition site; gly-pro rich domains; and the tyrosine kinase catalytic domain, or region of DDR1-FPPAPWWPPGPPPTNFSSLELEPRGQQPVAKAEGSPT (380-416 amino acids). Antibodies raised against this unique peptide segment will be specific for mammalian DDR1 receptor only. For therapeutic purposes, antibodies may be conjugated to cytotoxic moieties, including radioactive isotopes (radionuclides), chemotoxic agents such as differentiation inducers and small chemotoxic drugs, toxin proteins, and derivatives thereof. It is demonstrated that antibodies binding to extracellular sequences of DDR1 are internalized, thereby providing a mechanism for such cytotoxic moieties to kill targeted tumor cells.

In another embodiment of the invention, a binding member specific for DDR1 is a DDR1 ligand or binding fragment derived therefrom, including fibronectin, collagen, and a soluble fragment of DDR1 capable of homotypic binding. Such binding members may be conjugated to a cytotoxic moiety.

Formulations of DDR1 targeted therapeutic agents, e.g. specific binding members including antibodies and other ligands; small molecules that bind and/or inhibit DDR1; small molecules that bind and/or inhibit DDR1 signalling, mechanism based inhibitors of DDR1 tyrosine kinase; and the like, may be administered to brain tumor patients in a form stabilized for stability and retention in the brain region. The formulation may comprise one, two or more DDR1 directed therapeutic agents, and may further comprise additional therapeutic agents targeted to a different brain tumor target protein. The therapeutic formulation may be administered in combination with surgical treatment of the tumor, including pre-surgical treatment, administration at the time of surgery, or as a follow-up to surgery. The therapeutic formulation may be administered in combination with a chemotherapeutic agent or other targeted therapeutic agents. The DDR1 targeted therapeutic agents are effective in inhibiting the invasion of glioma cells, including astrocytomas grade II and grade III and grade IV tumors; and can result can result in inhibition of cellular functions, involving cell adhesion, cell-cell interaction, cell proliferation, cell survival, migration, invasion and angiogenesis. Therapeutic molecules can result in inhibiting structural and signaling functions, display anti-angiogenic properties, inhibit proliferation and migration and tumor growth, thus demonstrating a role as a diagnostic and therapeutic agent in vascular and cancer biology.

### BRIEF DESCRIPTION OF THE DRAWINGS

FIGS. 1A and 1B are blots of normal human brain tissue samples probed for expression of DDR1. FIG. 1A is a Northern Blot, and FIG. 1B, is a graphical representation measuring relative intensities for DDR1 mRNA expression in different regions of the brain as shown in FIG. 1A.

FIGS. 2A and 2B are Western blots of human Glioma derived cell lines and tissue. This figure shows an exprsssion profile for DDR1 isoforms in glioma cells. A C-terminal antibody was used and DDR1 cleaved C-terminal fragment can be detected.

FIGS. 3A and 3B are immunohistochemical analyses of normal brain and glioma tissue, demonstrating tumor specific expression of DDR1.

FIG. 4A-4B shows DDR1 promotes migration (4A) and invasion into basement membrane matrix by glioma cells (4B).

FIG. 5 demonstrates that DDR1 overexpresion in U87 cells promote increased presence of MMP-2 (pro and active MMP-2). Increased levels of MMP-1 and MMP-9 are also seen.

FIG. 6 is a graph depicting the viability of cells that overexpress a DDR1 extracellular domain construct.

FIG. 7 is a Western Blot showing autophosphorylation and processing of DDR1 protein upon ligand stimulation. DDR1 is phosphorylated by Type 1 Collagen, Fibronectin and EGF.

FIG. 8 is a bar graph quantification of DDR1 ligand induced internalization.

FIGS. 9A-9B. DDR1 expression profile. (A) Immunohistochemical analysis of normal brain and glioblastoma tissue demonstrate tumor specific expression of DDR1. IHC sections stained with DDR1 polyclonal antibody show very low levels of expression in normal brain sections (a-c) compared to glioblastoma tissues (d-f) and Astrocytoma Grade III sections (g-i). The experiments were repeated two additional times with similar results. Original magnification, 20.×. (B) Immunoblot analysis of lysates of glioma cells in culture and tissue samples from glioma, normal brain, lung and liver, and probed with polyclonal anti-DDR1 Ab.

FIGS. 10A-10B. Expression and phosphorylation of DDR1a, DDR1b in G140 glioma cell lines. (A) Cell lysates from G140 cells, mock, DDR1a and DDR1b in G140-expressing cell lines were analyzed by Western blot by using an anti-DDR1 antibody. (B) DDR1 is tyrosine phosphorylated in response to Collagen type 1 (10 μg/ml). At the indicated times, cells were lysed and DDR1 proteins were immunoprecipitated. The level of tyrosine phosphorylation of DDR1 in the immunoprecipitate was determined by western blotting using 4G10 phosphotyrosine antibody (Upper Panel). The level of DDR1 was detected by probing with the DDR1 antibody (Lower Panel).

FIGS. 11A-3D. Overexpression of DDR1a stimulates glioma Migration and Invasion. (A) Migration of parental G140 cells or those expressing DDR1a, DDR1b, or vector (mock) was evaluated. Cells adhering to the underside of the membrane coated with Collagen type 1 (B) or Matrigel (C) were stained with calcein and fluorescence was measured. The number of G140 cells expressing DDR1a displayed increased migration (A) and invasion (B and C) compared with G140, G140-DDR1b, and mock cells. The data are shown as means: bars+/−SD. The assays were performed in triplicate three independent times. *, P<0.005, significant migration and invasion compared to G140. G140-DDR1b and mock cells. (D) Anti-DDR1 antibody (mab-48B3-20 μg/ml) inhibited G140-DDR1a stimulated cell invasion through matrigel. Vehicle and mouse IgG (20 μg/ml) were included as controls. *, P<0.005, significant inhibition compared to G140-DDR1a cells incubated with mouse IgG.

FIGS. 12A-12B. DDR1a overexpression and MMP production. (A) Conditioned media was collected and analyzed for MMP production from parental G140 cells or those expressing vector (mock), DDR1a, or DDR1b. (B) Similarly, conditioned media was collected and analyzed for MMP production from the cell lines following stimulation with collagen type 1 (10 μg/ml). MMP-9, pro-MMP2, intermediate MMP-2 and active MMP-2 bands are indicated. Overexpression of DDR1a in G140 cells stimulates the activation of MMP-2.

FIGS. 13A-13C. DDR1a stimulates glioma cell invasion through activation of MMP-2 in G140 cells. (A) Conditioned media from G140 cells overexpressing DDR1a was analyzed for MMP activity following collagen 1 stimulation and in the presence or absence of the MMP inhibitor GM6001 (25 μm). GM6001 inhibited the activation of pro-MMP-2 in G140-DDR1a cells. Invasion through Collagen 1 (B) or Matrigel (C) by parental G140 cells or those expressing DDR1a, DDR1b, or vector (mock) was evaluated in the presence of GM60001 (25 μM). GM60001 inhibited DDR1a mediated invasion of G140 cells through both Collagen type 1 (B) and Matrigel (C) coated membranes. The data are shown as means: bars+/−SD. The assays were performed in triplicate three independent times. *, P<0.005, significant inhibition compared to non-treated G140-DDR1a cells.

FIG. 14A-14C. Adhesion to collagen type 1 in DDR1-overexpressing glioma cells. (A) Time dependent adhesion of parental G140 cells or those expressing DDR1a, DDR1b, or vector (mock) was evaluated at 0, 30, 60, 90 and 120 min. A time dependent increase in adherence to Collagen 1 was seen with both G140-DDR1a and G140-DDR1b cells when compared to parental and mock cell lines. (B and C) Adhesion of parental G140 cells or those expressing DDR1a, DDR1b, or vector (mock) was evaluated in the presence of antibody. The indicated cell lines were incubated with vehicle, anti-β1 integrin (b1-10 μg/ml), anti-DDR1 (10 μg/ml) antibodies, or mgG (10 μg/ml) for 1 hr. The cells were then plated onto Collagen type-1 (10 μg/ml) or BSA (10 μg/ml) coated wells. After 60 min (B) and 120 min (C) of incubation, the number of attached cells was determined. The data are shown as means: bars+/−SD. The assays were performed in triplicate three independent times. *, P<0.005, significant inhibition compared to non-treated G140-DDR1a and G140-DDR1b cells.

### DETAILED DESCRIPTION OF THE EMBODIMENTS

Differential cloning between cancerous and normal brains has identified the brain tumor target gene DDR1 by DNA sequence analysis. The upregulation of this protein in high grade astrocytoma is important because it provides a specific marker for neoplastic cells, and is expected to mediate the initiation and progression of brain tumors. Inhibition of the gene and/or protein activity can be advantageous in treating brain tumors. DDR1 provides a target for immunotherapeutic agents that either deliver cytotoxic agents to directly promote tumor cell death, or that alter the function of the brain tumor protein targets to inhibit the normal physiology of the tumor cell. In addition, immunoimaging agents targeted to the brain tumor protein targets can be utilized to visualize the tumor mass in diagnostic methods, e.g. magnetic resonance imaging (MRI), radiography, etc. and/or in surgery, e.g. by the use of optically visual dye moieties in an immunoimaging agent, etc.

Therapeutic and prophylactic treatment methods for individuals suffering, or at risk of brain tumor, involve administering either a therapeutic or prophylactic amount of an agent that inhibits DDR1, or that specifically binds to DDR1.

In one embodiment of the invention DDR1 expression is used as a specific marker for the diagnosis and treatment of gliomas. Included in such methods is DDR1 of the DDR1a isotype, of the DDR1b isotype, of the DDR1d and DDR1e isotype, the cleaved C-terminal and soluble fragments of DDR1, e.g. cleaved at the RFRR protease recognition site.

### Disease Conditions

The present methods are applicable to brain tumors, particularly glioblastoma. In general, the goals of brain tumor treatments are to remove as many tumor cells as possible, e.g. with surgery, kill as many of the cells left behind after surgery as possible with radiation and/or chemotherapy, and put remaining tumor cells into a nondividing, quiescent, non-invasive state for as long as possible with radiation and chemotherapy. Careful imaging surveillance is a crucial part of medical care, because tumor regrowth requires alteration of current treatment, or, for patients in the observation phase, restarting treatment.

Brain tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors. Agents that bind to, or otherwise inhibit DDR1 function can inhibit the invasiveness of glioblastoma cells, and are effective in preventing the spread of brain tumors through collagen and basement membranes. Inhibitors can also target matrix metalloproteases that are induced by activation of DDR1, e.g. thiol, alkylcarbonyl, phosponamidate and hydroxamate MMP inhibitor compounds, such as marimastat and prinomastat.

Glioblastoma multiforme is the most malignant stage of astrocytoma, with survival times of less than 2 years for most patients. Histologically, these tumors are characterized by dense cellularity, high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

There are biologic subsets of astrocytomas, which may reflect the clinical heterogeneity observed in these tumors. These subsets include brain stem gliomas, which are a form of pediatric diffuse, fibrillary astrocytoma that often follow a malignant course. Brain stem GBMs share genetic features with those adult GBMs that affect younger patients. Pleomorphic xanthoastrocytoma (PXA) is a superficial, low-grade astrocytic tumor that predominantly affects young adults. While these tumors have a bizarre histological appearance, they are typically slow-growing tumors that may be amenable to surgical cure. Some PXAs, however, may recur as GBM. Pilocytic astrocytoma is the most common astrocytic tumor of childhood and differs clinically and histopathologically from the diffuse, fibrillary astrocytoma that affects adults. Pilocytic astrocytomas do not have the same genomic alterations as diffuse, fibrillary astrocytomas. Subependymal giant cell astrocytomas (SEGA) are periventricular, low-grade astrocytic tumors that are usually associated with tuberous sclerosis (TS), and are histologically identical to the so-called “candle-gutterings” that line the ventricles of TS patients. Similar to the other tumorous lesions in TS, these are slowly-growing and may be more akin to hamartomas than true neoplasms. Desmoplastic cerebral astrocytoma of infancy (DCAI) and desmoplastic infantile ganglioglioma (DIGG) are large, superficial, usually cystic, benign astrocytomas that affect children in the first year or two of life.

Oligodendrogliomas and oligoastrocytomas (mixed gliomas) are diffuse, usually cerebral tumors that are clinically and biologically most closely related to the diffuse, fibrillary astrocytomas. The tumors, however, are far less common than astrocytomas and have generally better prognoses than the diffuse astrocytomas. Oligodendrogliomas and oligoastrocytomas may progress, either to WHO grade III anaplastic oligodendroglioma or anaplastic oligoastrocytoma, or to WHO grade IV GBM. Thus, the genetic changes that lead to oligodendroglial tumors constitute yet another pathway to GBM.

Ependymomas are a clinically diverse group of gliomas that vary from aggressive intraventricular tumors of children to benign spinal cord tumors in adults. Transitions of ependymoma to GBM are rare. Choroid plexus tumors are also a varied group of tumors that preferentially occur in the ventricular system, ranging from aggressive supratentorial intraventricular tumors of children to benign cerebellopontine angle tumors of adults. Choroid plexus tumors have been reported occasionally in patients with Li-Fraumeni syndrome and von Hippel-Lindau (VHL) disease.

Medulloblastomas are highly malignant, primitive tumors that arise in the posterior fossa, primarily in children. Meningiomas are common intracranial tumors that arise in the meninges and compress the underlying brain. Meningiomas are usually benign, but some “atypical” meningiomas may recur locally, and some meningiomas are frankly malignant and may invade the brain or metastasize. Atypical and malignant meningiomas are not as common as benign meningiomas. Schwannomas are benign tumors that arise on peripheral nerves. Schwannomas may arise on cranial nerves, particularly the vestibular portion of the eighth cranial nerve (vestibular schwannomas, acoustic neuromas) where they present as cerebellopontine angle masses. Hemangioblastomas are tumors of uncertain origin that are composed of endothelial cells, pericytes and so-called stromal cells. These benign tumors most frequently occur in the cerebellum and spinal cord of young adults. Multiple hemangioblastomas are characteristic of von Hippel-Lindau disease (VHL). Hemangiopericytomas (HPCs) are dural tumors which may display locally aggressive behavior and may metastasize. The histogenesis of dural-based hemangiopericytoma (HPC) has long been debated, with some authors classifying it as a distinct entity and others classifying it as a subtype of meningioma.

The symptoms of both primary and metastatic brain tumors depend mainly on the location in the brain and the size of the tumor. Since each area of the brain is responsible for specific functions, the symptoms will vary a great deal. Tumors in the frontal lobe of the brain may cause weakness and paralysis, mood disturbances, difficulty thinking, confusion and disorientation, and wide emotional mood swings. Parietal lobe tumors may cause seizures, numbness or paralysis, difficulty with handwriting, inability to perform simple mathematical problems, difficulty with certain movements, and loss of the sense of touch. Tumors in the occipital lobe can cause loss of vision in half of each visual field, visual hallucinations, and seizures. Temporal lobe tumors can cause seizures, perceptual and spatial disturbances, and receptive aphasia. If a tumor occurs in the cerebellum, the person may have ataxia, loss of coordination, headaches, and vomiting. Tumors in the hypothalamus may cause emotional changes, and changes in the perception of hot and cold. In addition, hypothalamic tumors may affect growth and nutrition in children. With the exception of the cerebellum, a tumor on one side of the brain causes symptoms and impairment on the opposite side of the body.

### DDR1

DDR1 was identified in brain tumors by creating cDNA libraries from glioblastoma tissues. The cDNA's from control and disease states were subjected to kinetic re-annealing hybridization during which normalization of transcript abundances and enrichment for differentially expressed transcripts (i.e., subtraction) occurs. Only clones displaying a significant transcriptional induction and/or repression were sequenced and carried forward for expression profiling, using a variety of temporal, spatial and disease-related probe sets. Selected clones showing a significant transcriptional induction and/or repression were sequenced and functionally annotated in a proprietary database structure (See WO01/13105). Because large sequence fragments were utilized in the sequencing step, the data generated has a much higher fidelity and specificity than other approaches, such as SAGE. The resulting sequence information was compared to public databases using the BLAST (blastn) and iterative-Smith Waterman analysis for protein sequence comparisons.

TABLE 1

| | | NUCLEOTIDE | SEQ | PROTEIN | |
|---|---|---|---|---|---|
| AGY ID | DESCRIPTION | ACCESSION | ID | ACCESSION | SEQ ID |
| AL00003_CP2_K01 | Homo sapiens discoidin domain receptor | NM_001954 | 1 | NP_001945 | 2 |
| | family, member 1 (DDR1), transcript variant 2 | | | | |
| AL00003_CP2_K01 | Homo sapiens discoidin domain receptor | NM_013993 | 3 | NP_054699 | 4 |
| | family, member 1 (DDR1), transcript variant 1 | | | | |
| AL00003_CP2_K01 | Homo sapiens discoidin domain receptor | NM_013994 | 5 | NP_054700 | 6 |
| | family, member 1 (DDR1), transcript variant 3 | | | | |

DDR1 is a 913 amino acid (125 kd) cell surface receptor. Upon collagen activation, it is phosphorylated and proteolytically processed. The protein features include a signal sequence (SEQ ID NO:1, residues 1-18); a discoidin domain involved in collagen binding and collagen induced receptor dimerization (SEQ ID NO:1, residues 34-107); an F5/8 type C domain involved in cell surface carbohydrate binding (SEQ ID NO:1, residues 31-185); an RFRR protease recognition site (SEQ ID NO:1, residues 304-307); the stalk region, which undergoes structural changes following receptor binding and dimerization and transmits signal resulting in transphosphorylation of the kinase domain (SEQ ID NO:1, residues 199-412); gly-pro rich domains important in ligand or substrate binding (SEQ ID NO:1, residues 377415 and 476-601); a transmembrane domain (SEQ ID NO:1, residues 417-443); and a tyrosine kinase catalytic domain (SEQ ID NO:1, residues 610-905).

DDR1 is activated by collagen type I to type VI, DDR2 is only activated by fibrillar collagens. The 160 aa long discoidin domain essential for collagen binding is followed by a 200 aa long stalk region. Both regions are important for receptor signaling. DDR1 may also bind to fibronectin, and may form homotypic dimers. The extracellular domain expressing cells show a decrease in proliferation thereby indicating that the soluble 52 kd extracellular DDR1 form may bind cells and act as a ligand.

In order to identify regions (epitopes) in the extracellular domain of human DDR1 that are targets for specific antibodies, the extracellular region of human DDR1 (residues 1-416) was used as a query to find orthologs or paralogs in protein and nucleic acid databases. The search identified orthologs in rat and mouse. The paralog, DDR2, was identified in human, hamster and mouse, and showed significant similarity over the entire extracellular part of DDR1. Multiple alignment of the extracellular region of human, rat and mouse DDR1, as well as human, hamster and mouse DDR2 shows that DDR1s deviate most significantly from DDR2s in the C-terminal part of the extracellular region. Multiple alignment of the extracellular parts of human, rat, and mouse DDR1 shows that there is significant conservation throughout the extracellular region, including the region where they deviate most from DDR2s. In the latter region there is a long stretch of amino acids that are 100% identical in rat, mouse and human: FPPAPWWPPGPPPTNFSSLELEPRGQQPVAKAEGSPT (SEQ ID NO:1, residues 380-416). This sequence was found to have a match only with DDR1, no significant similarity was observed with any other mammalian protein. Therefore, antibodies raised against this peptide segment are specific for mammalian DDR1 receptor, and would not cross-react with DDR2 receptor nor with any other mammalian proteins. This region is also C-terminal to the furin-cleavage site.

DDR1 appears in multiple isoforms, including: a, b, c, d and e, which are generated by alternative splicing. DDR1b contains an additional 37 amino acids, which is present in the juxtamembrane region. The DDR1c-isoform contains additional 6 amino acids at the beginning of the kinase domain between exons 13 and 14 and is the longest isoform. The DDR1a isoform lacks exon 11. Deletions of exon 11 and 12 gives rise to isoform DDR1d. During rat post natal development, the amount of DDR1b considerably increases in comparison to the DDR1a isoform. In DDR1e isoform, the first half of exon 10 and exons 11 and 12 are missing. DDR1d and DDR1e are kinase dead mutants. DDR1 is partially processed into a 63-kd membrane anchored DDR1b-subunit and a soluble 54 kd DDR1a-subunit by an unidentified protease.

Sequences of the DDR1 isoforms or publicly available, for example at Genbank:

| transcript | Genbank accession number |
|---|---|
| DDR1.a | AL528663 |
| DDR1.b | BG116520 |
| DDR1.c | BI036228 |
| DDR1.d | BE899403 |
| DDR1.e | BG696424 |
| DDR1.f | BC008716 |
| DDR1.gk | L11315 |
| DDR1.j | BI597388 |
| DDR1.k | AL537189 |
| DDR1.l | NM_013993 |
| DDR1.m | NM_001954 |
| DDR1.n | Z29093 |
| DDR1.o | L20817 |
| DDR1.p | L57508 |
| DDR1.q | BI458024 |
| DDR1.r | AF353182 |
| DDR1.s | AF353183 |

DDR1 is expressed mainly in epithelial cells of human mammary gland, kidney, lung, colon, thyroid, brain and islets of langerhans. DDR1b protein is the predominant isoform expressed during embryogenesis, whereas the a-isoform is upregulated in certain mammary carcinoma cell lines. The longest isoform is DDR1c. DDR1a promotes migration of leukocytes in three-dimensional collagen lattices. Among three DDR1 isoforms (a, b, and c), DDR1a was the major transcript in leukocytes. Overexpression of either DDR1a or DDR1b resulted in an increase in adherence in these cells. However, only DDR1a, but not DDR1b, over-expressing cells exhibited marked pseudopod extension and migrated successfully through three-dimensional collagen lattices. DDR1 also has been shown to control growth and adhesion of mesangial cells.

Two novel isoforms of DDR1, DDR1d and DDR1e have been identified from human colon carcinoma cells. Both new isoforms have been predicted to be membrane anchored but kinase-deficient receptors. The alternative splicing event takes place in the juxtamembrane region, which contains sequence motifs essential for the interaction with cellular substrates and regulatory proteins. Based on their structure, receptors with mutated or deleted kinase domain have been proposed to act as suppressors of full-length, enzymatic active receptors by forming heterodimers and blocking signaling in a dominant negative manner. However, DDR1d and DDR1e do not influence collagen-mediated DDR1 signaling. A role in cell adhesion, or sequestering and presenting collagen as ligand to the DDR1 full-length receptor has been postulated. These novel DDR1 isoforms may also have a role during embryogenesis and tumor progression.

Studies with smooth muscle cells (SMCs) from wild-type and DDR1(−/−) mice has shown that tyrosine kinase activity of discoidin domain receptor 1 is necessary for smooth muscle cell migration and matrix metalloproteinase expression. DDR1(−/−) SMCs exhibited impaired attachment to and migration toward a type I collagen substrate. These results suggest that phosphorylation of DDR1 kinase is important for cell migration.

Identification of genes in the DDR1 signaling pathway may be performed through physical association of gene products, or through database identification of known physiologic pathways. Among the methods for detection protein-protein association are co-immunoprecipitation, crosslinking and co-purification through gradients or chromatographic columns. The two-hybrid system detects the association of proteins in vivo, as described by Chien et al. (1991) Proc. Natl. Acad. Sci. USA 88:9578-9582. The two-hybrid system or related methodology may be used to screen activation domain libraries for proteins that interact with a known “bait” gene protein.

Functional validation is useful in determining whether the gene plays a role in tumor initiation, progression or maintenance. The term “functional validation” as used herein refers to a process whereby one determines whether modulation of expression or function of a candidate gene or set of such genes causes a detectable change in a cellular activity or cellular state for a reference cell, which cell can be a population of cells such as a tissue or an entire organism. The detectable change or alteration that is detected can be any activity carried out by the reference cell. Specific examples of activities or states in which alterations can be detected include, but are not limited to, phenotypic changes (e.g., cell morphology, cell proliferation, cell viability and cell death); cells acquiring resistance to a prior sensitivity or acquiring a sensitivity which previously did not exist; protein/protein interactions; cell movement; intracellular or intercellular signaling; cell/cell interactions; cell activation; release of cellular components (e.g., hormones, chemokines and the like); and metabolic or catabolic reactions.

A variety of options are available for functionally validating candidate genes. Such methods as RNAi technology can be used. Antisense technology can also be utilized to functionally validate a candidate gene. In this approach, an antisense polynucleotide that specifically hybridizes to a segment of the coding sequence for the candidate gene is administered to inhibit expression of the candidate gene in those cells into which it is introduced. The functional role that a candidate gene plays in a cell can also be assessed using gene “knockout” approaches in which the candidate gene is deleted, modified, or inhibited on either a single or both alleles. The cells or animals can be optionally be reconstituted with a wild-type candidate gene as part of a further analysis.

In one embodiment of the invention, RNAi technology is used in functional validation. As used herein, RNAi technology refers to a process in which double-stranded RNA is introduced into cells expressing a candidate gene to inhibit expression of the candidate gene, i.e., to “silence” its expression. The dsRNA is selected to have substantial identity with the candidate gene. In general such methods initially involve transcribing a nucleic acids containing all or part of a candidate gene into single- or double-stranded RNA. Sense and anti-sense RNA strands are allowed to anneal under appropriate conditions to form dsRNA. The resulting dsRNA is introduced into reference cells via various methods and the degree of attenuation in expression of the candidate gene is measured using various techniques. Usually one detects whether inhibition alters a cellular state or cellular activity. The dsRNA is prepared to be substantially identical to at least a segment of a candidate gene. Because only substantial sequence similarity between the gene and the dsRNA is necessary, sequence variations between these two species arising from genetic mutations, evolutionary divergence and polymorphisms can be tolerated. Moreover, the dsRNA can include various modified or nucleotide analogs. Usually the dsRNA consists of two separate complementary RNA strands. However, in some instances, the dsRNA may be formed by a single strand of RNA that is self-complementary, such that the strand loops back upon itself to form a hairpin loop. Regardless of form, RNA duplex formation can occur inside or outside of a cell.

A number of options are available to detect interference of candidate gene expression (i.e., to detect candidate gene silencing). In general, inhibition in expression is detected by detecting a decrease in the level of the protein encoded by the candidate gene, determining the level of mRNA transcribed from the gene and/or detecting a change in phenotype associated with candidate gene expression.

### Compound Screening

DDR1 protein sequences are used in screening of candidate compounds, including antibodies and small organic molecules, for the ability to bind to and/or inhibit DDR1 protein activity. Agents that inhibit DDR1 proteins are of interest as therapeutic agents for the treatment of brain tumors. Such compound screening may be performed using an in vitro model, a genetically altered cell or animal, or purified protein corresponding to DDR1 protein or a fragment thereof. One can identify ligands or substrates that bind to, modulate or mimic the action of the encoded polypeptide.

Polypeptides useful in screening include those encoded by the DDR1 gene, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to DDR1, or a homolog or variant thereof.

Transgenic animals or cells derived therefrom are also used in compound screening. Transgenic animals may be made through homologous recombination, where the normal locus corresponding to DDR1 is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in enzymatic activity, oncogenesis, signal transduction, etc. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development, for example by overexpressing in neural cells. By providing expression of the target protein in cells in which it is not normally produced, one can induce changes in cell behavior.

Compound screening identifies agents that modulate function of DDR1. Of particular interest are screening assays for agents that have a low toxicity for normal human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function of DDR1 protein. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of phosphatase inhibitors can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, all of which are incorporated by reference herein in their entirety.)

A “combinatorial library” is a collection of compounds in which the compounds comprising the collection are composed of one or more types of subunits. Methods of making combinatorial libraries are known in the art, and include the following: U.S. Pat. Nos. 5,958,792; 5,807,683; 6,004,617; 6,077,954; which are incorporated by reference herein. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules that are different from each other. Each of the different molecules is present in a detectable amount. The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, hydrocarbon substituents, e.g. aliphatic, alicyclic substituents, aromatic, aliphatic and alicyclic-substituted aromatic nuclei, and the like, as well as cyclic substituents; substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like); and hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Candidate agents of interest also include peptides and derivatives thereof, e.g. high affinity peptides or peptidomimetic substrates for DDR1 that is an enzyme or transporter, particularly a substrate modified to act as an inhibitor. DDR1 is a tyrosine kinase and mechanism based inhibitors include analogs having tyrosine residues replaced with an inhibitory analog, see Liljebris et al. (2002) Bioorg Med Chem 10(10):3197-212; Liljebris et al. (2002) J Med Chem 45(9):1785-98; and Jia et al. (2001) J Med Chem 44(26):4584-94.

Generally, peptide agents encompassed by the methods provided herein range in size from about 3 amino acids to about 100 amino acids, with peptides ranging from about 3 to about 25 being typical and with from about 3 to about 12 being more typical. Peptide agents can be synthesized by standard chemical methods known in the art (see, e.g., Hunkapiller et al., Nature 310:105-11, 1984; Stewart and Young, Solid Phase Peptide Synthesis, 2nd Ed., Pierce Chemical Co., Rockford, Ill., (1984)), such as, for example, an automated peptide synthesizer. In addition, such peptides can be produced by translation from a vector having a nucleic acid sequence encoding the peptide using methods known in the art (see, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein).

Peptide libraries can be constructed from natural or synthetic amino acids. For example, a population of synthetic peptides representing all possible amino acid sequences of length N (where N is a positive integer), or a subset of all possible sequences, can comprise the peptide library. Such peptides can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. Nonclassical amino acids or chemical amino acid analogs can be used in substitution of or in addition into the classical amino acids. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methy1 amino acids, C α-methyl amino acids, N α-methyl amino acids, and amino acid analogs in general. Furthermore, the amino acid can be D (dextrorotary) or L (levorotary).

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to DDR1, as at least some of the compounds so identified are likely inhibitors. The binding assays usually involve contacting DDR1 with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89.

Certain screening methods involve screening for a compound that modulates the expression of DDR1. Such methods generally involve conducting cell-based assays in which test compounds are contacted with one or more cells expressing DDR1 and then detecting and an increase in expression. Some assays are performed with tumor cells that express endogenous DDR1. Other expression assays are conducted with non-neuronal cells that express an exogenous DDR1 gene.

The level of expression or activity can be compared to a baseline value. As indicated above, the baseline value can be a value for a control sample or a statistical value that is representative of expression levels for a control population. Expression levels can also be determined for cells that do not express DDR1, as a negative control. Such cells generally are otherwise substantially genetically the same as the test cells. Various controls can be conducted to ensure that an observed activity is authentic including running parallel reactions with cells that lack the reporter construct or by not contacting a cell harboring the reporter construct with test compound. Compounds can also be further validated as described below.

Compounds that are initially identified by any of the foregoing screening methods can be further tested to validate the apparent activity. The basic format of such methods involves administering a lead compound identified during an initial screen to an animal that serves as a model for humans and then determining if a DDR1 gene is in fact upregulated. The animal models utilized in validation studies generally are mammals. Specific examples of suitable animals include, but are not limited to, primates, mice, and rats.

Active test agents identified by the screening methods described herein that inhibit DDR1 protein activity and/or tumor growth can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

### Pharmaceutical Formulations

Formulations of DDR1 targeted therapeutic agents, e.g. specific binding members including antibodies and other ligands; small molecules that bind and/or inhibit DDR1; mechanism based inhibitors of DDR1 tyrosine kinase; and the like, may be administered to brain tumor patients in a form stabilized for stability and retention in the brain region. The formulation may comprise one, two or more DDR1 directed therapeutic agents, and may further comprise additional therapeutic agents targeted to a different brain tumor target protein. The therapeutic formulation may be administered in combination with surgical treatment of the tumor, including pre-surgical treatment, administration at the time of surgery, or as a follow-up to surgery. The DDR1 targeted therapeutic agents are effective in inhibiting the invasion of glioblastoma cells, including astrocytomas grade II and grade III tumors; and can result in the necrosis of tumor cells.

One strategy for drug delivery through the blood brain barrier (BBB) entails disruption of the BBB, either by osmotic means such as mannitol or leukotrienes, or biochemically by the use of vasoactive substances such as bradykinin, or surgical methods to directly introduce the agent. The potential for using BBB opening to target specific agents to brain tumors is also an option. A BBB disrupting agent can be co-administered with the therapeutic or imaging compositions of the invention when the compositions are administered by intravascular injection. Other strategies to go through the BBB may entail the use of endogenous transport systems, including carrier-mediated transporters such as glucose and amino acid carriers, receptor-mediated transcytosis for insulin or transferrin, and active efflux transporters such as p-glycoprotein. Active transport moieties may also be conjugated to the therapeutic or imaging compounds for use in the invention to facilitate transport across the epithelial wall of the blood vessel. Alternatively, drug delivery behind the BBB is by intrathecal delivery of therapeutics or imaging agents directly to the cranium, as through an Ommaya reservoir.

Depending on the strategy for the therapeutic, the formulation can be placed into several catagories. For example, antibody formulations include native antibody, armed antibody (coupled to isotope, or toxin), or heterobifunctional antibody (genetically engineered, or T-cell attractant). Armed antibodies (isotope or toxin conjugated) are generally given intracavitary after the resection of either a primary or a recurrent tumor, as long as the ventricles are not opened. The method is based on the overexpression of the target in the intracranial compartment with little or no crossreaction elsewhere in the brain. All present trials work with local intracavitary or intratumoral application. Heterobifunctional antibodies are designed to bind to the cell surface and then attract T-cells into the tumor with their other arm to elicit an immune response. This method of delivery is reserved for a local application either into a cavity or the tissue itself.

Formulations, e.g. antibody formulations, may be optimized for retention and stabilization in the brain. When the agent is administered into the cranial compartment, it is desirable for the agent to be retained in the compartment, and not to diffuse or otherwise cross the blood brain barrier. Stabilization techniques include enhancing the size of the antibody, by cross-linking, multimerizing, or linking to groups such as polyethylene glycol, polyacrylamide, neutral protein carriers, etc. in order to achieve an increase in molecular weight.

Other strategies for increasing retention include the entrapment of the agent in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Administration

In the context of Glioma therapy several treatment situations are possible: the tumor may be removed and therapeutic agent administered after surgery; the tumor may be present and a therapeutic agent added to treat the tumor mass or part thereof; the tumr may recur and the therapeutic agent added to treat the recurrent mass; and the recurrent tumor may be removed and the therapeutic agent added into the cavity.

Surgery is usually the first step in treating most brain tumors. The object of most brain tumor surgeries is to remove or reduce as much of its bulk as possible. By reducing the size, other therapies, particularly radiotherapy, can be more effective. The goals of surgery are: 1) to remove as much of the tumor as possible so there will be less of a tumor burden for adjuvant therapies, 2) to provide tumor tissue for microscopic examination in order to reach an exact diagnosis in order to guide additional treatment, and 3) to provide direct access to the malignant tumor cells for other treatments, such as implants for gene therapy. If surgical removal is not immediately feasible or if the tumor is inaccessible, that is, in an area of the brain that is deep and inoperable, then a stereotaxic biopsy may be performed to establish a diagnosis. This is a minimally invasive procedure whereby computer guidance allows a probe to reach almost any area of the brain through a small hole in the skull.

The standard procedure is called craniotomy where the neurosurgeon removes a piece of skull bone to expose the area of brain over the tumor. The tumor is located and then removed. The surgeon has various surgical options for breaking down and removing the tumor, including standard surgical procedures; laser microsurgery (which produces great heat and vaporizes tumor cells); ultrasonic aspiration (which uses ultrasound to break the glioma tumor into small pieces, which are then suctioned out); etc.

Special techniques have been developed to allow maximum removal of tumor while protecting healthy brain cells. For example, stereotaxy has become a useful adjunct to both surgery (stereotactic surgery) and radiotherapy (stereotactic radiotherapy). Cortical localization, or stimulation, uses a probe that passes a tiny electrical current to delicately stimulate a specific area of the brain. This produces a visible response of the body part (such as a twitch in a leg), which the stimulated region of the brain controls. The surgeon then knows to avoid those areas during the operation. Image guided surgery uses a three-dimensional picture of the patients' brain derived from computed tomography (CT) or magnetic resonance imaging (MRI) scans. The image, with various views of the brain, is displayed on a monitor in the operating room. During surgery, as the surgeon's instrument touches a part of the brain, a camera sends the image to a computer, which calculates the position of the surgical tool and displays it in its proper location on the 3-D image. The surgeon then can look at the monitor and see what structures to avoid. Neurosurgeons are also investigating the use of a technique in which external magnetic fields direct a magnet-tipped flexible catheter to the tumor site through a path that avoids areas of the brain that could cause harm.

The compositions of the invention may be administered using any medically appropriate procedure, e.g., intravascular (intravenous, intraarterial, intracapillary) administration, injection into the cerebrospinal fluid, intracavity or direct injection in the tumor. Intrathecal administration maybe carried out through the use of an Ommaya reservoir, in accordance with known techniques. (F. Balis et al., Am J. Pediatr. Hematol. Oncol. 11, 74, 76 (1989). For the imaging compositions of the invention, administration via intravascular injection is preferred for pre-operative visualization of the tumor. Post-operative visualization or visualization concurrent with an operation may be through intrathecal or intracavity administration, as through an Ommaya reservoir, or also by intravascular administration.

One method for administration of the therapeutic compositions of the invention is by deposition into the inner cavity of a cystic tumor by any suitable technique, such as by direct injection (aided by stereotaxic positioning of an injection syringe, if necessary) or by placing the tip of an Ommaya reservoir into a cavity, or cyst, for administration. Where the tumor is a solid tumor, the antibody may be administered by first creating a resection cavity in the location of the tumor. This procedure differs from an ordinary craniotomy and tumor resection only in a few minor respects. As tumor resection is a common treatment procedure, and is often indicated to relieve pressure, administration of the therapeutic compositions of the invention can be performed following tumor resection. Following gross total resection in a standard neurosurgical fashion, the cavity is preferable rinsed with saline until all bleeding is stopped by cauterization. Next the pia-arachnoid membrane, surrounding the tumor cavity at the surface, is cauterized to enhance the formation of fibroblastic reaction and scarring in the pia-arachnoid area. The result is the formation of an enclosed, fluid-filled cavity within the brain tissue at the location from where the tumor was removed. After the cyst has been formed, either the tip of an Ommaya reservoir or a micro catheter, which is connected to a pump device and allows the continuous infusion of an antibody solution into the cavity, can be placed into the cavity. See, e.g., U.S. Pat. No. 5,558,852, incorporated fully herein by reference.

Alternatively, a convection-enhanced delivery catheter may be implanted directly into the tumor mass, into a natural or surgically created cyst, or into the normal brain mass. Such convection-enhanced pharmaceutical composition delivery devices greatly improve the diffusion of the composition throughout the brain mass. The implanted catheters of these delivery devices utilize high-flow microinfusion (with flow rates in the range of about 0.5 to 15.0 μl/minute), rather than diffusive flow, to deliver the therapeutic or imaging composition to the brain and/or tumor mass. Such devices are described in U.S. Pat. No. 5,720,720, incorporated fully herein by reference.

The effective amount of a therapeutic composition to be given to a particular patient will depend on a variety of factors, several of which will be different from patient to patient. A competent clinician will be able to determine an effective amount of a therapeutic agent to administer to a patient to retard the growth and promote the death of tumor cells, or an effective amount of an imaging composition to administer to a patient to facilitate the visualization of a tumor. Dosage of the antibody-conjugate will depend on the treatment of the tumor, route of administration, the nature of the therapeutics, sensitivity of the tumor to the therapeutics, etc. Utilizing LD₅₀ animal data, and other information available for the conjugated cytotoxic or imaging moiety, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Imaging moieties are typically less toxic than cytotoxic moieties and may be administered in higher doses in some embodiments. Utilizing ordinary skill, the competent clinician will be able to optimize the dosage of a particular therapeutic or imaging composition in the course of routine clinical trials.

The compositions can be administered to the subject in a series of more than one administration. For therapeutic compositions, regular periodic administration (e.g., every 2-3 days) will sometimes be required, or may be desirable to reduce toxicity. For therapeutic compositions that will be utilized in repeated-dose regimens, antibody moieties which do not provoke immune responses are preferred.

To test the efficacy in an in vivo model of intratumoral application, a guidescrew system may be used, which allows the placement of a tumor cell deposit into a defined spot intracranially and the development of tumor at that spot. Thereafter, this spot can be targeted repeatedly with injections through this screw which is fixed in the skull and is hollow to guide an injection needle. This allows a lengthy treatment schedule and the application of large molecules which otherwise would not get to the tumor.

### Combination Therapies

Brain tumors tend to be heterogeneous in character, and pervasive throughout the brain tissue. This combination often makes them difficult to treat. In some cases, it may be preferred to use various combinations of therapeutic agents, in order to more fully target all of the cells exhibiting tumorigenic characteristics. Combinations of interest include administration of a DDR1 inhibitor in conjunction with chemotherapeutic agents, and/or with chemosensitizers. Chemotherapeutic agents are known in the art, and include, for example, include alkylating agents, such as nitrogen mustards, e.g. mechlorethamine, cyclophosphamide, melphalan (L-sarcolysin), etc.; and nitrosoureas, e.g. carmustine (BCNU), lomustine (CCNU), semustine (methyl-CCNU), streptozocin, chlorozotocin, etc. Antimetabolite agents include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc. Other chemotherapeutic agents include azathioprine; brequinar; alkaloids and synthetic or semi-synthetic derivatives thereof, e.g. vincristine, vinblastine, vinorelbine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithrmycin); anthracenediones, e.g. mitoxantrone; azirinopyrrolo indolediones, e.g. mitomycin; and the like. Other chemotherapeutic agents include metal complexes, e.g. cisplatin (cis-DDP), carboplatin, etc.; ureas, e.g. hydroxyurea; and hydrazines, e.g. N-methylhydrazine.

Another combination of interest is the administration of a DDR1 inhibitor in combination with radiation, and/or with radiation sensitizers. Radiosensitizers are compounds that, when combined with radiation, produce greater tumor cell kill than expected from a simple additive effect. Sensitizers include metronidazole, misonidazole, etanidazole, taxol, 5 fluorouracil, hydroxyurea, angiogenesis inhibitors, protein kinase C inhibitors, compounds such as motexafin gadolinium, and the like. Alternatively, the DDR1 inhibitor may act as a sensitizing agent.

Radiation therapy for brain tumors is widely used, and will typically be used in combination with administration of a radiosensitizer. For example, ionizing radiation from X-rays or gamma rays may be delivered from an external source. Another technique for delivering radiation to cancer cells is internal radiotherapy, which places radioactive implants directly in the tumor so that the radiation dose is concentrated in a small area.

Antibodies may be formulated against DDR1, or may be formulated as a cocktail comprising antibodies reactive against two or more targets, where the targets may comprise DDR1 in combination with other brain tumor targets, e.g. PTPζ, Class II MHC antigens, RPTP, etc.

Such combination treatments may administer a DDR1 inhibitor with a second agent, and administering the blended therapeutic to the patient as described. The skilled administering physician will be able to take such factors as combined toxicity, and individual agent efficacy, into account when administering such combined agents. Additionally, those of skill in the art will be able to screen for potential cross-reaction with each other, in order to assure full efficacy of each agent.

Alternatively, several individual brain tumor protein target compositions may be administered simultaneously or in succession for a combined therapy. This may be desirable to avoid accumulated toxicity from several reagents, or to more closely monitor potential adverse reactions to the individual reagents. Thus, cycles such as where a therapeutic agent is administered on day one, followed by a second on day two, then a period with out administration, followed by re-administration of the therapeutics on different successive days, is comprehended within the present invention. Another combination therapy could include that of a small molecule drug and an antibody therapeutic against the individual brain tumor protein targets that are described in U.S. Pat. No. 6,455,026, and co-pending patent application Ser. Nos. 10/328,544; 10/329,258; 09/983,000; 60/369,743; 60/369,991; 60/369,985; 60/378,588; and 60/452,169, incorporated fully herein by reference.

### Nucleic Acids

The sequences of DDR1 genes find use in diagnostic and therapeutic methods, for the recombinant production of the encoded polypeptide, and the like. The nucleic acids of the invention include nucleic acids having a high degree of sequence similarity or sequence identity to a DDR1 coding sequence. Sequence identity can be determined by hybridization under stringent conditions, for example, at 50° C. or higher and 0.1×SSC (9 mM NaCl/0.9 mM Na citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to a DDR1 sequence under stringent hybridization conditions. Further specific guidance regarding the preparation of nucleic acids is provided by Fleury et al. (1997) Nature Genetics 15:269-272; Tartaglia et al., PCT Publication No. WO 96/05861; and Chen et al., PCT Publication No. WO 00/06087, each of which is incorporated herein in its entirety.

A suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression in tumor cells.

Probes specific to DDR1 can be generated using the provided nucleic acid sequences. The probes are preferably at least about 18 nt, 25 nt, 50 nt or more of the corresponding contiguous sequence a provided sequence, and are usually less than about 2, 1, or 0.5 kb in length. Preferably, probes are designed based on a contiguous sequence that remains unmasked following application of a masking program for masking low complexity. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

### Polypeptides

DDR1 polypeptides are of interest for screening methods, as reagents to raise antibodies, as therapeutics, and the like. Such polypeptides can be produced through isolation from natural sources, recombinant methods and chemical synthesis. In addition, functionally equivalent polypeptides may find use, where the equivalent polypeptide may contain deletions, additions or substitutions of amino acid residues that result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity as a DDR1 polypeptide.

The polypeptides may be produced by recombinant DNA technology using techniques well known in the art. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

Typically, the coding sequence is placed under the control of a promoter that is functional in the desired host cell to produce relatively large quantities of the gene product. An extremely wide variety of promoters are well-known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

In mammalian host cells, a number of viral-based expression systems may be used, including retrovirus, lentivirus, adenovirus, adeno-associated virus, and the like. In cases where an adenovirus is used as an expression vector, the coding sequence of interest can be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing differentially expressed or pathway gene protein in infected hosts.

Specific initiation signals may also be required for efficient translation of the genes. These signals include the ATG initiation codon and adjacent sequences. In cases where a complete gene, including its own initiation codon and adjacent sequences, is inserted into the appropriate expression vector, no additional translational control signals may be needed. However, in cases where only a portion of the gene coding sequence is inserted, exogenous translational control signals must be provided. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

For long-term, high-yield production of recombinant proteins, stable expression is preferred. For example, cell lines that stably express the differentially expressed or pathway gene protein may be engineered. Rather than using expression vectors that contain viral origins of replication, host cells can be transformed with DNA controlled by appropriate expression control elements, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the target protein. Such engineered cell lines may be particularly useful in screening and evaluation of compounds that affect the endogenous activity of the DDR1 protein. A number of selection systems may be used, including but not limited to the herpes simplex virus thymidine kinase, hypoxanthine-guanine phosphoribosyltransferase, and adenine phosphoribosyltransferase genes. Antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

The polypeptide may be labeled, either directly or indirectly. Any of a variety of suitable labeling systems may be used, including but not limited to, radioisotopes such as ¹²⁵I; enzyme labeling systems that generate a detectable colorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by a Fab expression library.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)).

As an option to recombinant methods, polypeptides and oligopeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of a DDR1 protein can be synthesized and then joined together. Methods for conducting such reactions are described by Grant (1992) Synthetic Peptides: A User Guide, W.H. Freeman and Co., N.Y.; and in “Principles of Peptide Synthesis,” (Bodansky and Trost, ed.), Springer-Verlag, Inc. N.Y., (1993).

For various purposes, for example as an immunogen, the entire DDR1 polypeptide or a fragment derived therefrom may be used. Preferably, one or more 8-30 amino acid peptide portions, e.g. of an extracellular domain may be utilized, with peptides in the range of 10-20 being a more economical choice. Regions of interest include the sequence FPPAPWWPPGPPPTNFSSLELEPRGQQPVAKAEGSPT (SEQ ID NO:1, residues 380-416); the discoidin domain (SEQ ID NO:1, residues 34-107); the F5/8 type C domain (SEQ ID NO:1, residues 31-185); the RFRR protease recognition site (SEQ ID NO:1, residues 304-307); the stalk region (SEQ ID NO:1, residues 199-412); gly-pro rich domains (SEQ ID NO:1, residues 377-415 and 476-601); and the tyrosine kinase catalytic domain (SEQ ID NO:1, residues 610-905). Custom-synthesized peptides in this range are available from a multitude of vendors, and can be conjugated to KLH or BSA. Alternatively, peptides in excess of 30 amino acids may be synthesized by solid-phase methods, or may be recombinantly produced in a suitable recombinant protein production system. In order to ensure proper protein glycosylation and processing, an animal cell system (e.g., Sf9 or other insect cells, CHO or other mammalian cells) is preferred.

### Specific Binding Members

The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used.

Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; lipid and lipid-binding protein; etc. The specific binding pairs may include analogs, derivatives and fragments of the original specific binding member. For example, a receptor and ligand pair may include peptide fragments, chemically synthesized peptidomimetics, labeled protein, derivatized protein, etc.

In another embodiment of the invention, a binding member specific for DDR1 is a DDR1 ligand or binding fragment derived therefrom, including fibronectin, collagen, and a soluble fragment of DDR1 capable of homotypic binding. Such binding members may be conjugated to a cytotoxic moiety.

In a preferred embodiment, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The term includes monoclonal antibodies, multispecific antibodies (antibodies that include more than one domain specificity), human antibody, humanized antibody, and antibody fragments with the desired biological activity.

Antibodies that bind specifically to one of the brain tumor protein targets are referred to as α(DDR1). The specific or selective fit of a given structure and its specific epitope is sometimes referred to as a “lock and key” fit. The archetypal antibody molecule is the immunoglobulin, and all types of immunoglobulins, IgG, e.g. IgG1, IgG2a, IgG2b, IgG3, IgG4, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, a DDR1 antigen comprising an antigenic portion of the polypeptide is initially injected into any of a wide variety of mammals (e.g., mice, rats, rabbits, sheep or goats). When utilizing an entire protein, or a larger section of the protein, antibodies may be raised by immunizing the production animal with the protein and a suitable adjuvant (e.g., Fruend's, Fruend's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the brain tumor protein target, a superior immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, 1975 Nature 256:495 (the disclosures of which are hereby incorporated by reference).

Large quantities of monoclonal antibodies from the secreting hybridomas may then be produced by injecting the clones into the peritoneal cavity of mice and harvesting the ascites fluid therefrom. The mice, preferably primed with pristane, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones, which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Preferably, recombinant antibodies are produced in a recombinant protein production system that correctly glycosylates and processes the immunoglobulin chains, such as insect or mammalian cells. An advantage to using insect cells, which utilize recombinant baculoviruses for the production of antibodies, is that the baculovirus system allows production of mutant antibodies much more rapidly than stably transfected mammalian cell lines. In addition, insect cells have been shown to correctly process and glycosylate eukaryotic proteins, which prokaryotic cells do not. Finally, the baculovirus expression of foreign protein has been shown to constitute as much as 50-75% of the total cellular protein late in viral infection, making this system an excellent means of producing milligram quantities of the recombinant antibodies.

Antibodies with a reduced propensity to induce a violent or detrimental immune response in humans (such as anaphylactic shock), and which also exhibit a reduced propensity for priming an immune response which would prevent repeated dosage with the antibody therapeutic or imaging agent are preferred for use in the invention. Even through the brain is relatively isolated behind the blood brain barrier, an immune response still can occur in the form of increased leukocyte infiltration, and inflammation. Although some increased immune response against the tumor is desirable, the concurrent binding and inactivation of the therapeutic or imaging agent generally outweighs this benefit. Thus, humanized, single chain, chimeric, or human antibodies, which produce less of an immune response when administered to humans, are preferred for use in the present invention. Also included in the invention are multi-domain antibodies, and anti-idiotypic antibodies that “mimic” DDR1. For example, antibodies that bind to a DDR1 domain and competitively inhibit the binding of DDR1 to its ligand may be used to generate anti-idiotypes that “mimic” DDR1 and, therefore, bind, activate, or neutralize a DDR1, DDR1 ligand, DDR1 receptor, or DDR1 ligand. Such anti-idiotypic antibodies or Fab fragments of such anti-idiotypes can be used in therapeutic regimens involving a DDR1 mediated pathway (see, for example, Greenspan and Bona (1993) FASEB J 7(5):437-444; Nissinoff (1991) J. Immunol. 147(8):2429-2438.

A chimeric antibody is a molecule in which different portions are derived from different animal species, for example those having a variable region derived from a murine mAb and a human immunoglobulin constant region. Techniques for the development of chimeric antibodies are described in the literature. See, for example, Morrison et al. (1984) Proc. Natl. Acad. Sci. 81:6851-6855; Neuberger et al. (1984) Nature 312:604-608; Takeda et al. (1985) Nature 314:452-454. Single chain antibodies are formed by linking the heavy and light chain fragments of the Fv region via an amino acid bridge, resulting in a single chain polypeptide. See, for example, Huston et al., Science 242:423-426; Proc. Natl. Acad. Sci. 85:5879-5883; and Ward et al. Nature 341:544-546.

Antibody fragments that recognize specific epitopes may be generated by techniques well known in the field. These fragments include, without limitation, F(ab′)₂ fragments, which can be produced by pepsin digestion of the antibody molecule, and Fab fragments, which can be generated by reducing the disulfide bridges of the F(ab′)₂ fragments.

In one embodiment of the invention, a human or humanized antibody is provided, which specifically binds to the extracellular region of DDR1 with high affinity. Binding of the antibody to the extracellular region can lead to receptor down regulation or decreased biological activity, thereby decreasing cell proliferation, invasion and/or tumor size cell adhesion, migration and angiogenesis as biological functional activities. Low affinity binders may also be useful for some immuno-therapies. See Lonberg et al. (1994) Nature 368:856-859; and Lonberg and Huszar (1995) Internal Review of Immunology 13:65-93. In another aspect of the invention, a humanized antibody is provided that specifically binds to the extracellular region of DDR1 with high affinity, and which bears resemblance to the human antibody. These antibodies resemble human antibodies and thus can be administered to a human patient with minimal negative side effects.

Humanized antibodies are human forms of non-human antibodies. They are chimeras with a minimum sequence derived from of non-human Immunoglobulin. To overcome the intrinsic undesirable properties of murine monoclonal antibodies, recombinant murine antibodies engineered to incorporate regions of human antibodies, also called “humanized antibodies” are being developed. This alternative strategy was adopted as it is difficult to generate human antibodies directed to human antigens such as cell surface molecules. A humanized antibody contains complementarity determining region (CDR) regions and a few other amino acid of a murine antibody while the rest of the antibody is of human origin.

Chimeric antibodies may be made by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference). Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference.

Alternatively, polyclonal or monoclonal antibodies may be produced from animals that have been genetically altered to produce human immunoglobulins. Techniques for generating such animals, and deriving antibodies therefrom, are described in U.S. Pat. Nos. 6,162,963 and 6,150,584, incorporated fully herein by reference.

Alternatively, single chain antibodies (Fv, as described below) can be produced from phage libraries containing human variable regions. See U.S. Pat. No. 6,174,708. Intrathecal administration of single-chain immunotoxin, LMB-7 [B3(Fv)-PE38], has been shown to cure of carcinomatous meningitis in a rat model. Proc Natl. Acad. Sci USA 92, 2765-9, all of which are incorporated by reference fully herein.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Fv fragments are heterodimers of the variable heavy chain domain (V_{H}) and the variable light chain domain (V_{L}). The heterodimers of heavy and light chain domains that occur in whole IgG, for example, are connected by a disulfide bond. Recombinant Fvs in which V_{H} and V_{L} are connected by a peptide linker are typically stable, see, for example, Huston et al., Proc. Natl. Acad. Sci. USA 85:5879-5883 (1988) and Bird et al., Science 242:423-426 (1988), both fully incorporated herein, by reference. These are single chain Fvs which have been found to retain specificity and affinity and have been shown to be useful for imaging tumors and to make recombinant immunotoxins for tumor therapy. However, researchers have bound that some of the single chain Fvs have a reduced affinity for antigen and the peptide linker can interfere with binding. Improved Fv's have been also been made which comprise stabilizing disulfide bonds between the V_{H} and V_{L} regions, as described in U.S. Pat. No. 6,147,203, incorporated fully herein by reference. Any of these minimal antibodies may be utilized in the present invention, and those which are humanized to avoid HAMA reactions are preferred for use in embodiments of the invention.

In addition, derivatized immunoglobulins with added chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of the present invention. For convenience, the term “antibody” or “antibody moiety” will be used throughout to generally refer to molecules which specifically bind to an epitope of the brain tumor protein targets, although the term will encompass all immunoglobulins, derivatives, fragments, recombinant or engineered immunoglobulins, and modified immunoglobulins, as described above.

Candidate anti-DDR1 antibodies can be tested for by any suitable standard means, e.g. ELISA assays, etc. As a first screen, the antibodies may be tested for binding against the immunogen, or against the entire brain tumor protein target extracellular domain or protein. As a second screen, anti-DDR1 candidates may be tested for binding to an appropriate tumor cell line, or to primary tumor tissue samples. For these screens, the anti-DDR1 candidate antibody may be labeled for detection. After selective binding to the brain tumor protein target is established, the candidate antibody, or an antibody conjugate produced as described below, may be tested for appropriate activity (i.e., the ability to decrease tumor cell growth and/or to aid in visualizing tumor cells) in an in vivo model, such as an appropriate tumor cell line, or in a mouse or rat human brain tumor model, as described below. In a preferred embodiment, anti-DDR1 protein antibody compounds may be screened using a variety of methods in vitro and in vivo. These methods include, but are not limited to, methods that measure binding affinity to a target, biodistribution of the compound within an animal or cell, or compound mediated cytotoxicity. These and other screening methods known in the art provide information on the ability of a compound to bind to, modulate, or otherwise interact with the specified target and are a measure of the compound's efficacy.

Antibodies that alter the biological activity of DDR1 protein may be assayed in functional formats, such as astrocytoma cell culture or mouse/rat CNS tumor model studies. In astroctyoma cell models of activity, expression of the protein is first verified in the particular cell strain to be used. If necessary, the cell line may be stably transfected with a coding sequence of the protein under the control of an appropriate constituent promoter, in order to express the protein at a level comparable to that found in primary tumors. The ability of the astrocytoma cells to survive in the presence of the candidate function-altering anti-protein antibody is then determined. In addition to cell-survival assays, cell invasion assays and cell adhesion assays may be utilized to determine the effect of the candidate antibody therapeutic agent on the tumor-like behavior of the cells. Alternatively, if DDR1 is involved in angiogenesis, assays may be utilized to determine the ability of the candidate antibody therapeutic to inhibit vascular neogenesis, an important function in tumor biology.

The binding affinity of the DDR1 antibody may be determined using Biacore SPR technology, as is known in the art. In this method, a first molecule is coupled to a Dextran CM-5 sensor chip (Pharmacia), and the bound molecule is used to capture the antibody being tested. The antigen is then applied at a specific flow rate, and buffer applied at the same flow rate, so that dissociation occurs. The association rate and dissociation rates and corresponding rate constants are determined by using BIA evaluation software. For example, see Malmqvist (1993) Surface plasmon resonance for detection and measurement of antibody-antigen affinity and kinetics. Volume: 5:282-286; and Davies (1994) Nanobiology 3:5-16. Sequential introduction of antibodies permits epitope mapping. Once the antigen has been introduced, the ability of a second antibody to bind to the antigen can be tested. Each reactant can be monitored individually in the consecutive formation of multimolecular complexes, permitting multi-site binding experiments to be performed.

The binding of some ligands to their receptors can result in receptor-mediated internalization. This property may be desirable, e.g. with antibody therapeutics such as immunoliposomes; or undesirable, e.g. with antibody directed enzyme-prodrug therapy (ADEPT), where the enzyme needs to be present at the cell surface to convert non active prodrugs into active cytotoxic molecules.

Similarly, in vivo models for human brain tumors, particularly nude mice/SCID mice model or rat models, have been described, for example see Antunes et al. (2000). J Histochem Cytochem 48, 847-58; Price et al. (1999) Clin Cancer Res 5, 845-54; and Senner et al. (2000). Acta Neuropathol (Berl) 99, 603-8. Once correct expression of the protein in the tumor model is verified, the effect of the candidate anti-protein antibodies on the tumor masses in these models can be evaluated, wherein the ability of the anti-protein antibody candidates to alter protein activity is indicated by a decrease in tumor growth or a reduction in the tumor mass. Thus, antibodies that exhibit the appropriate anti-tumor effect may be selected without direct knowledge of the particular biomolecular role of the protein in oncogenesis. In vivo models may also be used to screen small molecule modulators of DDR1 function.

### Antibody Conjugates

The anti-DDR1 antibodies for use in the present invention may have utility without conjugation when the native activity of DDR1 is altered in the tumor cell. Such antibodies, which may be selected as described above, may be utilized without as a therapeutic agent. In another embodiment of the invention, DDR1 specific antibodies, which may or may not alter the activity of the target polypeptide, are conjugated to cytotoxic or imaging agents, which add functionality to the antibody.

The anti-DDR1 antibodies can be coupled or conjugated to one or more therapeutic cytotoxic or imaging moieties. As used herein, “cytotoxic moiety” is a moiety that inhibits cell growth or promotes cell death when proximate to or absorbed by the cell. Suitable cytotoxic moieties in this regard include radioactive isotopes (radionuclides), chemotoxic agents such as differentiation inducers and small chemotoxic drugs, toxin proteins, and derivatives thereof. “Imaging moiety” (I) is a moiety that can be utilized to increase contrast between a tumor and the surrounding healthy tissue in a visualization technique (e.g., radiography, positron-emission tomography, magnetic resonance imaging, direct or indirect visual inspection). Thus, suitable imaging moieties include radiography moieties (e.g. heavy metals and radiation emitting moieties), positron emitting moieties, magnetic resonance contrast moieties, and optically visible moieties (e.g., fluorescent or visible-spectrum dyes, visible particles, etc.). It will be appreciated by one of ordinary skill that some overlap exists between therapeutic and imaging moieties. For instance ²¹²Pb and ²¹²Bi are both useful radioisotopes for therapeutic compositions, but are also electron-dense, and thus provide contrast for X-ray radiographic imaging techniques, and can also be utilized in scintillation imaging techniques.

In general, therapeutic or imaging agents may be conjugated to the anti-DDR1 moiety by any suitable technique, with appropriate consideration of the need for pharmokinetic stability and reduced overall toxicity to the patient. A therapeutic agent may be coupled to a suitable antibody moiety either directly or indirectly (e.g. via a linker group). A direct reaction between an agent and an antibody is possible when each possesses a functional group capable of reacting with the other. For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity may also facilitate the use of moieties, or functional groups on moieties, which otherwise would not be possible.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups may be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), may be employed as a linker group. Coupling may be effected, for example, through amino groups, carboxyl groups, sulfhydryl groups or oxidized carbohydrate residues. There are numerous references describing such methodology, e.g., U.S. Pat. No. 4,671,958. As an alternative coupling method, cytotoxic or imaging moieties may be coupled to the anti-DDR1 antibody moiety through a an oxidized carbohydrate group at a glycosylation site, as described in U.S. Pat. Nos. 5,057,313 and 5,156,840. Yet another alternative method of coupling the antibody moiety to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the cytotoxic or imaging moiety.

Where a cytotoxic moiety is more potent when free from the antibody portion of the immunoconjugates of the present invention, it may be desirable to use a linker group that is cleavable during or upon internalization into a cell, or which is gradually cleavable over time in the extracellular environment. A number of different cleavable linker groups have been described. The mechanisms for the intracellular release of a cytotoxic moiety agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

Two or more cytotoxic and/or imaging moieties may be conjugated to an antibody, where the conjugated moieties are the same or different. By poly-derivatizing the anti-DDR1 antibody, several cytotoxic strategies can be simultaneously implemented; an antibody may be made useful as a contrasting agent for several visualization techniques; or a therapeutic antibody may be labeled for tracking by a visualization technique. Immunoconjugates with more than one moiety may be prepared in a variety of ways. For example, more than one moiety may be coupled directly to an antibody molecule, or linkers, which provide multiple sites for attachment (e.g., dendrimers) can be used. Alternatively, a carrier with the capacity to hold more than one cytotoxic or imaging moiety can be used.

A carrier may bear the cytotoxic or imaging moiety in a variety of ways, including covalent bonding either directly or via a linker group, and non-covalent associations. Suitable covalent-bond carriers include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier may also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful for imaging moiety conjugation to anti-DDR1 antibody moieties for use in the invention, as a sufficient amount of the imaging moiety (dye, magnetic resonance contrast reagent, etc.) for detection may be more easily associated with the antibody moiety. In addition, encapsulation carriers are also useful in chemotoxic therapeutic embodiments, as they can allow the therapeutic compositions to gradually release a chemotoxic moiety over time while concentrating it in the vicinity of the tumor cells.

Carriers and linkers specific for radionuclide agents (both for use as cytotoxic moieties or positron-emission imaging moieties) include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis. Such chelation carriers are also useful for magnetic spin contrast ions for use in magnetic resonance imaging tumor visualization methods, and for the chelation of heavy metal ions for use in radiographic visualization methods.

Preferred radionuclides for use as cytotoxic moieties are radionuclides that are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. Iodine and astatine isotopes are more preferred radionuclides for use in the therapeutic compositions of the present invention, as a large body of literature has been accumulated regarding their use. ¹³¹I is particularly preferred, as are other β-radiation emitting nuclides, which have an effective range of several millimeters. ¹²³I, ¹²⁵I, ¹³¹I, or ²¹¹At may be conjugated to antibody moieties for use in the compositions and methods utilizing any of several known conjugation reagents, including Iodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope may be utilized in the recited iodo-reagents. Radionuclides can be conjugated to anti-DDR1 antibody moieties by suitable chelation agents known to those of skill in the nuclear medicine arts.

Preferred chemotoxic agents include small-molecule drugs such as carboplatin, cisplatin, vincristine, taxanes such as paclitaxel and doceltaxel, hydroxyurea, gemcitabine, vinorelbine, irinotecan, tirapazamine, matrilysin, methotrexate, pyrimidine and purine analogs, and other suitable small toxins known in the art. Preferred chemotoxin differentiation inducers include phorbol esters and butyric acid. Chemotoxic moieties may be directly conjugated to the anti-DDR1 antibody moiety via a chemical linker, or may encapsulated in a carrier, which is in turn coupled to the anti-DDR1 antibody moiety.

Chemotherapy is helpful in controlling high-grade gliomas. A common combination of chemotherapeutics is “PCV”, which refers to the three drugs: Procarbazine, CCNU, and Vincristine. Temozolomide (Temodar) is approved by the FDA for treatment of anaplastic astrocytoma, and this drug is now widely used for high-grade gliomas. Neupogen may be administered to patients whose white blood counts fall to very low levels after chemotherapy.

Preferred toxin proteins for use as cytotoxic moieties include ricins A and B, abrin, diphtheria toxin, bryodin 1 and 2, momordin, trichokirin, cholera toxin, gelonin, Pseudomonas exotoxin, Shigella toxin, pokeweed antiviral protein, and other toxin proteins known in the medicinal biochemistry arts. The nontoxic ricin B chain is the moiety that binds to cells while the A chain is the toxic portion that inactivates protein synthesis—but only after delivery to the cytoplasm by the disulfide-linked B chain which binds to galactose-terminal membrane proteins. Abrin, diphtheria toxin, and Pseudomonas exotoxins all have similar 2-chain components; with one chain mediating cell membrane binding and entry and the toxic enzymatic A chain. Cholera has a pentameric binding subunit coupled to the toxic A chain. As these toxin agents may elicit undesirable immune responses in the patient, especially if injected intravascularly, it is preferred that they be encapsulated in a carrier for coupling to the anti-DDR1 antibody moiety.

Preferred radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Examples of such compositions which may be utilized for x-ray radiography are described in U.S. Pat. No. 5,709,846, incorporated fully herein by reference. Such moieties may be conjugated to the anti-DDR1 antibody moiety through an acceptable chemical linker or chelation carrier. In addition, radionuclides which emit radiation capable of penetrating the scull may be useful for scintillation imaging techniques. Suitable radionuclides for conjugation include ⁹⁹Tc, ¹¹¹In, and ⁶⁷Ga. Positron emitting moieties for use in the present invention include ¹⁸F, which can be easily conjugated by a fluorination reaction with the anti-DDR1 antibody moiety according to the method described in U.S. Pat. No. 6,187,284.

Preferred magnetic resonance contrast moieties include chelates of chromium(III), manganese(II), iron(II), nickel(II), copper(II), praseodymium(III), neodymium(III), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates may be conjugated to the anti-DDR1 antibody moieties through a suitable chemical linker.

Optically visible moieties for use as imaging moieties include fluorescent dyes, or visible-spectrum dyes, visible particles, and other visible labeling moieties. Fluorescent dyes such as ALEXA dyes, fluorescein, coumarin, rhodamine, bodipy Texas red, and cyanine dyes, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes include FDA-approved food dyes and colors, which are non-toxic, although pharmaceutically acceptable dyes which have been approved for internal administration are preferred. In preferred embodiments, such dyes are encapsulated in carrier moieties, which are in turn conjugated to the anti-DDR1 antibody. Alternatively, visible particles, such as colloidal gold particles or latex particles, may be coupled to the anti-DDR1 antibody moiety via a suitable chemical linker.

### Arrays

Arrays provide a high throughput technique that can assay a large number of polynucleotides in a sample. In one aspect of the invention, an array is constructed comprising DDR1 genes, proteins or antibodies in combination with other brain tumor targets, for example targets set forth in U.S. Pat. No. 6,455,026, and co-pending patent application Ser. Nos. 10/328,544; 10/329,258; 09/983,000; 60/369,743; 60/369,991; 60/369,985; 60/378,588; and 60/452,169, herein incorporated by reference.

This technology can be used as a tool to test for differential expression. Arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al., (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

The probes utilized in the arrays can be of varying types and can include, for example, synthesized probes of relatively short length (e.g., a 20-mer or a 25-mer), cDNA (full length or fragments of gene), amplified DNA, fragments of DNA (generated by restriction enzymes, for example) and reverse transcribed DNA. Both custom and generic arrays can be utilized in detecting differential expression levels. Custom arrays can be prepared using probes that hybridize to particular preselected subsequences of mRNA gene sequences or amplification products prepared from them.

Arrays can be used to, for example, examine differential expression of genes and can be used to determine gene function. For example, arrays can be used to detect differential expression of DDR1, where expression is compared between a test cell and control cell. Exemplary uses of arrays are further described in, for example, Pappalarado et al. (1998) Sem. Radiation Oncol. 8:217; and Ramsay. (1998) Nature Biotechnol. 16:40. Furthermore, many variations on methods of detection using arrays are well within the skill in the art and within the scope of the present invention. For example, rather than immobilizing the probe to a solid support, the test sample can be immobilized on a solid support which is then contacted with the probe. Additional discussion regarding the use of microarrays in expression analysis can be found, for example, in Duggan, et al., Nature Genetics Supplement 21:10-14 (1999); Bowtell, Nature Genetics Supplement 21:25-32 (1999); Brown and Botstein, Nature Genetics Supplement 21:33-37 (1999); Cole et al., Nature Genetics Supplement 21:38-41 (1999); Debouck and Goodfellow, Nature Genetics Supplement 21:48-50 (1999); Bassett, Jr., et al., Nature Genetics Supplement 21:51-55 (1999); and Chakravarti, Nature Genetics Supplement 21:56-60 (1999).

For detecting expression levels, usually nucleic acids are obtained from a test sample, and either directly labeled, or reversed transcribed into labeled cDNA. The test sample containing the labeled nucleic acids is then contacted with the array. After allowing a period sufficient for any labeled nucleic acid present in the sample to hybridize to the probes, the array is typically subjected to one or more high stringency washes to remove unbound nucleic acids and to minimize nonspecific binding to the nucleic acid probes of the arrays. Binding of labeled sequences is detected using any of a variety of commercially available scanners and accompanying software programs.

For example, if the nucleic acids from the sample are labeled with fluorescent labels, hybridization intensity can be determined by, for example, a scanning confocal microscope in photon counting mode. Appropriate scanning devices are described by e.g., U.S. Pat. No. 5,578,832 to Trulson et al., and U.S. Pat. No. 5,631,734 to Stern et al. and are available from Affymetrix, Inc., under the GeneChip™ label. Some types of label provide a signal that can be amplified by enzymatic methods (see Broude, et al., Proc. Natl. Acad. Sci. U.S.A. 91, 3072-3076 (1994)). A variety of other labels are also suitable including, for example, radioisotopes, chromophores, magnetic particles and electron dense particles.

Those locations on the probe array that are hybridized to labeled nucleic acid are detected using a reader, such as described by U.S. Pat. No. 5,143,854, WO 90/15070, and U.S. Pat. No. 5,578,832. For customized arrays, the hybridization pattern can then be analyzed to determine the presence and/or relative amounts or absolute amounts of known mRNA species in samples being analyzed as described in e.g., WO 97/10365.

### Diagnostic and Prognostic Methods

The differential expression of DDR1 in tumors indicates that this can serve as a marker for diagnosis, for imaging, as well as for therapeutic applications. In general, such diagnostic methods involve detecting an elevated level of expression of DDR1 gene transcripts or gene products in the cells or tissue of an individual or a sample therefrom including plasma, blood, CSF and other similar samples. A variety of different assays can be utilized to detect an increase in gene expression, including both methods that detect gene transcript and protein levels. More specifically, the diagnostic and prognostic methods disclosed herein involve obtaining a sample from an individual and determining at least qualitatively, and preferably quantitatively, the level of a DDR1 gene product expression in the sample. Usually this determined value or test value is compared against some type of reference or baseline value.

Nucleic acids or binding members such as antibodies that are specific for DDR1 are used to screen patient samples for increased expression of the corresponding mRNA or protein, or for the presence of amplified DNA in the cell. Samples can be obtained from a variety of sources. Samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from spinal fluid, or tumor biopsy samples. Also included in the term are derivatives and fractions of such cells and fluids. Samples can also be derived from in vitro cell cultures, including the growth medium, recombinant cells and cell components. Diagnostic samples are collected from an individual that has, or is suspected to have, a brain tumor. The presence of specific markers is useful in identifying and staging the tumor.

Nucleic Acid Screening Methods

Some of the diagnostic and prognostic methods that involve the detection of a DDR1 gene transcript begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample.

A number of methods are available for analyzing nucleic acids for the presence of a specific sequence, e.g. upregulated or downregulated expression. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp. 14.2-14.33.

A detectable label may be included in an amplification reaction. Suitable labels include fluorochromes, e.g. ALEXA dyes (available from Molecular Probes, Inc.); fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the walls of a microtiter well) and then permeabilizing the cells with an appropriate permeabilizing solution.

A solution containing labeled probes is then contacted with the cells and the probes allowed to hybridize. Excess probe is digested, washed away and the amount of hybridized probe measured. This approach is described in greater detail by Nucleic Acid Hybridization: A Practical Approach (Hames, et al., eds., 1987).

A variety of so-called “real time amplification” methods or “real time quantitative PCR” methods can also be utilized to determine the quantity of mRNA present in a sample. Such methods involve measuring the amount of amplification product formed during an amplification process. Fluorogenic nuclease assays are one specific example of a real time quantitation method that can be used to detect and quantitate transcripts. In general such assays continuously measure PCR product accumulation using a dual-labeled fluorogenic oligonucleotide probe—an approach frequently referred to in the literature simply as the “TaqMan” method. Additional details regarding the theory and operation of fluorogenic methods for making real time determinations of the concentration of amplification products are described, for example, in U.S. Pat. No. 5,210,015 to Gelfand, U.S. Pat. No. 5,538,848 to Livak, et al., and U.S. Pat. No. 5,863,736 to Haaland, each of which is incorporated by reference in its entirety.

Polypeptide Screening Methods

Various immunoassays designed to detect DDR1 isoforms may be used in screening. Detection may utilize staining of cells or histological sections, performed in accordance with conventional methods, using antibodies or other specific binding members that specifically bind to the DDR1 polypeptides. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the polypeptide corresponding to DDR1 in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding. After incubation, the insoluble support is generally washed of non-bound components. After washing, a solution containing a second antibody is applied. The antibody will bind to one of the proteins of interest with sufficient specificity such that it can be distinguished from other components present. The second antibodies may be labeled to facilitate direct, or indirect quantification of binding. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the targeted polypeptide, conveniently using a labeling method as described for the sandwich assay.

In some cases, a competitive assay will be used. In addition to the patient sample, a competitor to the targeted protein is added to the reaction mix. The competitor and the target compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

Imaging In Vivo

In some embodiments, the methods are adapted for imaging use in vivo, e.g., to locate or identify sites where tumor cells are present. In these embodiments, a detectably-labeled moiety, e.g., an antibody, which is specific for DDR1 is administered to an individual (e.g., by injection), and labeled cells are located using standard imaging techniques, including, but not limited to, magnetic resonance imaging, computed tomography scanning, and the like.

For diagnostic in vivo imaging, the type of detection instrument available is a major factor in selecting a given radionuclide. The radionuclide chosen must have a type of decay that is detectable by a given type of instrument. In general, any conventional method for visualizing diagnostic imaging can be utilized in accordance with this invention. Another important factor in selecting a radionuclide for in vivo diagnosis is that its half-life be long enough that it is still detectable at the time of maximum uptake by the target tissue, but short enough that deleterious radiation of the host is minimized. A currently used method for labeling with ^{99m}Tc is the reduction of pertechnetate ion in the presence of a chelating precursor to form the labile ^{99m}Tc-precursor complex, which, in turn, reacts with the metal binding group of a bifunctionally modified chemotactic peptide to form a ^{99m}Tc-chemotactic peptide conjugate.

The detectably labeled DDR1 specific antibody is used in conjunction with imaging techniques, in order to analyze the expression of the target. In one embodiment, the imaging method is one of PET or SPECT, which are imaging techniques in which a radionuclide is synthetically or locally administered to a patient. The subsequent uptake of the radiotracer is measured over time and used to obtain information about the targeted tissue. Because of the high-energy (γ-ray) emissions of the specific isotopes employed and the sensitivity and sophistication of the instruments used to detect them, the two-dimensional distribution of radioactivity may be inferred from outside of the body.

Among the most commonly used positron-emitting nuclides in PET are included ¹¹C, ¹³N, ¹⁵O, and ¹⁸F. Isotopes that decay by electron capture and/or γ emission are used in SPECT, and include ¹²³I and ^{99m}Tc.

### Modification of Gene Expression

Agents that modulate activity of DDR1 provide a point of therapeutic or prophylactic intervention, particularly agents that inhibit activity of the polypeptide, or expression of the gene. Numerous agents are useful in modulating this activity, including agents that directly modulate expression, e.g. expression vectors, antisense specific for the targeted polypeptide; and agents that act on the protein, e.g. specific antibodies and analogs thereof, small organic molecules that block catalytic activity, etc.

Methods can be designed to selectively deliver nucleic acids to certain cells. Examples of such cells include, neurons, microglia, astrocytes, endothelial cells, oligodendrocytes, etc. Certain treatment methods are designed to selectively express an expression vector to neuron cells and/or target the nucleic acid for delivery to CNS derived cells. One technique for achieving selective expression in nerve cells is to operably link the coding sequence to a promoter that is primarily active in nerve cells. Examples of such promoters include, but are not limited to, prion protein promoter, calcium-calmodulin dependent protein kinase promoter. Alternatively, or in addition, the nucleic acid can be administered with an agent that targets the nucleic acid to CNS derived cells. For instance, the nucleic acid can be administered with an antibody that specifically binds to a cell-surface antigen on the nerve cells or a ligand for a receptor on neuronal cells.

When liposomes are utilized, substrates that bind to a cell-surface membrane protein associated with endocytosis can be attached to the liposome to target the liposome to nerve cells and to facilitate uptake. Examples of proteins that can be attached include capsid proteins or fragments thereof that bind to nerve cells, antibodies that specifically bind to cell-surface proteins on nerve cells that undergo internalization in cycling and proteins that target intracellular localizations within CNS derived cells, (see, e.g., Wu et al. (1987) J. Biol. Chem. 262:4429-4432; and Wagner, et al. (1990) Proc. Natl. Acad. Sci. USA 87:3410-3414). Gene marking and gene therapy protocols are reviewed by Anderson et al. (1992) Science 256:808-813. Various other delivery options can also be utilized. For instance, a nucleic acid containing a sequence of interest can be injected directly into the cerebrospinal fluid. Alternatively, such nucleic acids can be administered by intraventricular injections.

Antisense molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Brain Tumors: Tumor tissue, confirmed as astrocytoma grade IV by neuropathology, from unknown patients was snap frozen in the operation hall and served as experimental sample. Human whole brain tissue (Clontech Laboratories, Palo Alto, USA) served as control sample. Poly-A⁺ RNA prepared from the cells was converted into double-stranded cDNA (dscDNA) and normalized as described in co-pending U.S. patent application Ser. No. 09/627,362, filed on Jul. 28, 2000. Subtractive hybridization was carried out using the dscDNA from tumors with an excess of dscDNA prepared from the same region of a non-cancerous brain. Differentially expressed gene fragments were cloned into a plasmid vector, and the resulting library was transformed into E. coli cells. Inserts of recombinant clones were amplified by the polymerase chain reaction (PCR). The PCR products (fragments of 200-2000 bp in size) were sequenced using an oligonucleotide complementary to common vector sequences. The resulting sequence information was compared to public databases using the BLAST (blastn) and Smith Waterman algorithm.

Quantitative Real Time PCR. Total RNA from normal brain tissue samples and tumor samples are isolated with Trizol (Gibco BRL) according to the manufacturer's instructions. SYBR Green real-time PCR amplifications are performed in an iCycler Real-Time Detection System (Bio-Rad Laboratories, Hercules, Calif.). The reactions are carried out in a 96-well plate in a 25-μl reaction volume containing 12.5 μl of 2×SYBR Green Master Mix (PE Applied Biosystems), a 0.9 μM concentration of each forward and reverse primer, 200 ng of total cDNA and supplemented to 25 μl with nuclease-free H₂O (Promega). Primers are designed using Primer3 developed by the Whitehead Institute for Biomedical Research and the primers (Operon Technologies, Alameda, Calif.) concentrations are optimized for use with the SYBR green PCR master mix reagents kit. The sizes of the amplicons are checked by running out the PCR product on a 1.5% agarose gel. The thermal profile for all SYBR Green PCRs is 50° C. for 2 minutes and 95° C. for 10 minutes, followed by 45 cycles of 95° C. for 15 seconds, 60° C. for 30 seconds followed by 72° C. for 40 seconds. The critical threshold cycle (Ct) is defined as the cycle at which the fluorescence becomes detectable above background and is inversely proportional to the logarithm of the initial number of template molecules. A standard curve is plotted for each primers set with Ct values obtained from amplification of 10-fold dilutions of cDNA obtained from whole brain. The standard curves are used to calculate the PCR efficiency of the primer set.

All PCR reactions are performed in duplicate. Quantification is performed using the comparative cycle threshold (CT) method, where CT is defined as the cycle number at which fluorescence reaches a set threshold value. The target transcript is normalized to an endogenous reference, and relative differences are calculated using the PCR efficiencies according to Pfaffl (Nucleic Acids Research, 2001). In order to demonstrate upregulation in tumor versus normal brain tissue, tumor samples and normal control brain tissue are surgically removed from various sources (including resection tissue, needle biopsy or other source of tissue). Total RNA is extracted from these samples using established methods and cDNA was generated for use in the real time quantitative PCR procedure.

Immunohistochemistry. For immunohistochemistry, human normal brain and tumor sections can be used. The human cancer tissue array slides are used to evaluate the tissue specific expression with antibodies. These paraffin embeded tissue array slides are dewaxed, washed in water and treated with target retrieval procedures. Alternatively, other sources of tumor and normal tissue can be analysed by immunohistochemistry, these include cryopreserved and needle biopsy material. Conventional immunhistochemical reactions are then carried out using an anti-DDR1 antibody. Tissue sections are analyzed using light microscopy to determine localization of staining, as well as intensity and tissue section ultrastructure. The protein expression level and localization of tumor proteins in tumor tissue are determined. These studies provide information on the staging and diagnosis of the brain tumor. In addition immunohistochemistry can be used to tailor therapy and determine treatment endpoints. Immunohistochemistry is also useful for screening anti-DDR1 antibodies.

Antigen Preparation and Immunizations: The quantity and quality of the antigen determines the number of mice immunized and the extent of the immune response. The antigen can include subdomains or regions of the target protein. Mice (transgenic or normal) are immunized in order to elicit and drive a high-affinity, and directed immune response. Lymphoid cells are recovered from immunized animals, and then arrayed and cultivated microtiter dishes as either immortalized (hybridomas) or primary B-cells.

Antibody Screening: The expanded populations of arrayed hybridomas or cultured B-cells are screened for antibodies that bind antigen using a variety of assay formats, for example by ELISA. The process is dependent on the number of positive results from a screen for gamma/kappa fusions and the nature of the antigen, typically it results in the identification of between 10 and 5,000 monoclonal antibodies. The antibodies can be bined by discrete epitope families; each of which is organized as a hierarchical continuum of kinetically ranked members. The antibodies that satisfy essential kinetic criteria, typically between 10 and 30, are advanced as leads for further evaluation.

Antibody Validation: Lead antibodies are validated in a battery of assays to assess the potencies of antibodies on the biology of the tumor target. These assays can be designed for naked (unarmed antibodies) or conjugated antibodies (armed with a toxin, or radioactive isotope or biotinylated) Assays used to characterize antibodies include affinity measures, internalization, blocking of ligands, immune response, activity in functional cellular assays, in vivo efficacy, in vivo toxicity, stability, and solubility.

Characterization of target antibodies for the ability to trigger internalization. Antibody-DDR1 complex internalization into glioma derived tumor cell is required for effective toxin immunoconjugate delivery. Measuring internalization of the antibody-brain tumor target complex demonstrates that a toxin-antibody conjugate can be specifically delivered to the tumor cells and allow effective tumor cell killing. Antibodies are screened for the ability to bind to the ectodomain of the tumor target and become internalized into human astrocytoma cells. The Cellomics Array Scan fluorescent microscope instrument is used to identify and quantitate internalized antibody-brain tumor complexes. Human glioma cells are plated onto black walled-clear bottom 96 well plates at a density of 25,000 cells per ml (2500 cells per well). The day after cell plating, the panel of antibodies is added onto the cells at concentrations from 0.1 ug/ml up to 100 ug/ml (5 doses, each in triplicate) and incubated for 0.5 h, and 2 h. Incubation of cells with antibodies is done at different time points.

Cells are rinsed once with HBSS and fixed with 3.7% neutral buffered formalin. The fixation solution is aspirated, and the plate washed with blocking buffer, and then incubated with permeabilization buffer for 10 minutes. The cells are then stained with fluorescence conjugated secondary antibody solution containing 10 ug/ml Hoechst 33342 (to stain nuclei) for 30 minutes at room temperature. Plates are washed twice, sealed, and stored in HBSS at 4 C. Images are acquired using the ArrayScan HCS system and internalized receptors quantitated using a proprietary algorithm. The algorithm measures the appearance and intensity of fluorescent receptor aggregates inside the cell. These measurements are represented as mean cytoplasmic intensity (amount of antibody-receptor complex inside the cell) and mean cytoplasmic texture (a measure of the endosome aggregates). Antibodies that trigger receptor internalization are further evaluated. Antibodies that bind to DDR1 on human glioma cells and become internalized are of particular interest. In some instances endocytosis serves as a surrogate marker for other therapeutic biologic effects, such as growth inhibition.

Characterization of DDR1 target antibodies in tumor cell growth assays. Gliomas are characterized as rapidly proliferating cells. Therefore antibodies are evaluated for the ability to inhibit glioma cell growth. The assay tests the effect of antibodies on the growth properties of cultured human glioma cells. Cells are seeded onto 96-well plates at a density of 3000-10,000 cells per well. The day after cell plating, the antibody is added onto the cells at concentrations from 0.1 ug/ml up to 100 ug/ml (5 doses, each in triplicate). Cells are grown with or without serum, in the presence or absence of ligand, in the presence or abscence of inhibitors (eg. MMP Inhibitors, MAPK Inhibitirs) to determine the effect of brain tumor target antibodies on cell growth and cell survival.

The effect of anti-DDR1 antibodies on glioma cell growth will be determined using a homogenous mix and read assay called Cell Titer Glo (Promega). This is a luminescence based assay that measures the level of ATP in the cell lysates. The more viable cells that are present, the greater the ATP level, and thereby stronger the luminescence signal. This reagent and the luminescence measurement is robust and convenient and antibody-mediated effects on cell adherence will not interfere with the readings (detached cells will not be washed off plate during processing). Antibodies that demonstrate cytotoxicity or inhibition of glioma cell growth are further characterized. This and similar assays (eg. BRDU assays) allow identification of a subset of antibodies that demonstrate efficacy in inhibiting glioma cell growth and cell survival.

Characterization of brain tumor target antibodies in tumor cell invasion assays: Local tumor cell invasiveness, which is a major morphological feature of gliomas, involves interactions between tumor cell and extracellular matrix, including adhesion, proteolysis, and migration of tumor cells through the locally modified microenvironment. This also invloves interaction between tumor cells and stromal cells. Therefore, anti-DDR1 antibodies can be evaluated for the ability to inhibit human glioma cell invasion and migration.

The assay is a quantitative determination of cell migration/invasion, evaluating the effect of brain tumor antibodies on the invasive properties of cultured human glioma cells. Human glioma cell lines are used to assess the ability of brain tumor target antibodies to inhibit cell invasion. The plates are coated with or without extracellular matrix solution prior to cell plating. Matrigel (BD Biosciences) is a mixture of extracellular matrix components that mimics a tumor microenvironment. In addition to matrigel, chambers will also be coated with different types of Collagen, Fibronectin and other extracellular matrices to study the role of DDR1 in invasion/migration. The cells are plated onto migration assay plates at a density of 10,000 to 25, 000 cells per well. (modified boyden chambers). Replicate sets of plates are used to measure different time points (eg, 0 h, 4 h and 24 h, 48 hrs). DDR1-antibodies at concentrations from 0.1 ug/ml up to 100 ug/ml (5 doses, each in triplicate) are added to the wells. DMEM with or without 5% serum was added to the lower chambers in the presence or absence of chemoattractant. After 0 h, 4 h and 24 h, 48 hrs, migrating/invading cells adhering to the underside of the membrane is stained with Calcein and fluorescence emitted by cells that have invaded is measured. Controlling Glioma cell invasion is an important characteristic of a brain tumor target therapeutic.

### Results

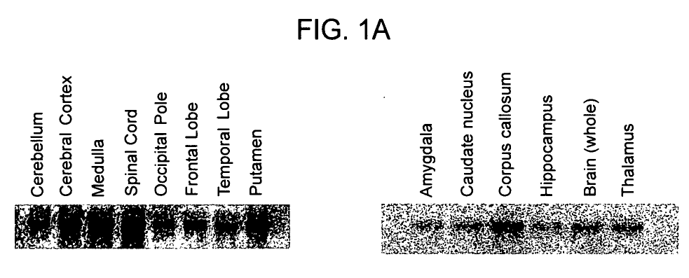

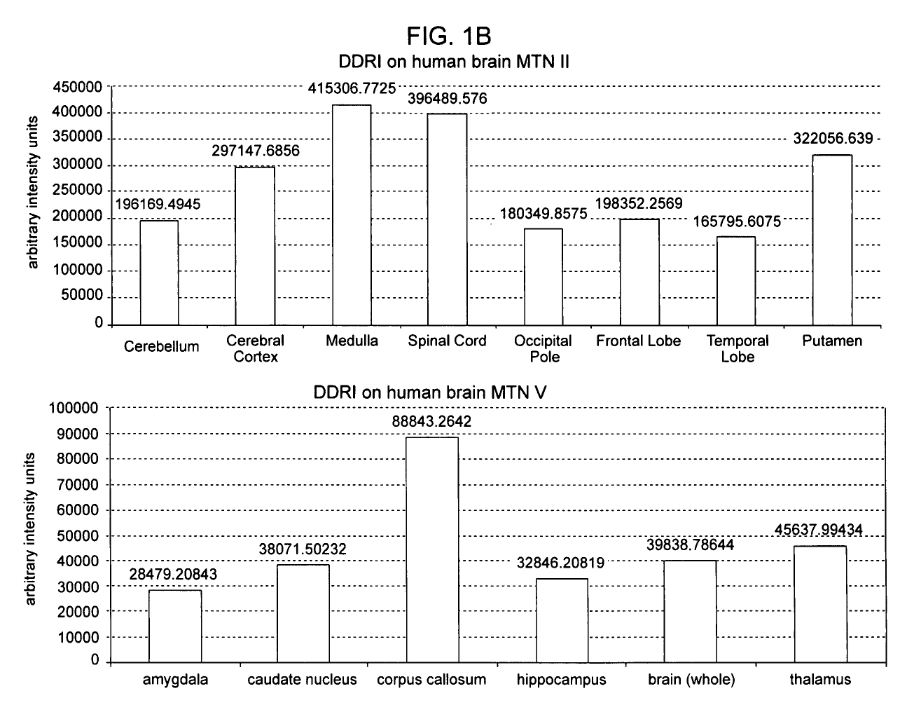

Expression of DDR1. Studies by Functional Genomics has demonstrated that the gene encoding DDR1 protein was upregulated in a panel of 14 high grade Glioma tumor samples by 1.7 fold increase (P value 2.07E-07). The expression of DDR1 mRNA in normal brain tissues was also examined by Northern Blot analysis, and the presence of DDR1 protein was tested by Western Blot analysis. As shown in FIG. 1A, DDR1 is expressed in various regions of the brain, including the corpus callosum, medulla and spinal cord. Northern Blot analysis revealed a single band at 4.4 kb. FIG. 1B measures the relative intensity of each band from the Northern blot in FIG. 1A. The blots were normalized for β-actin.

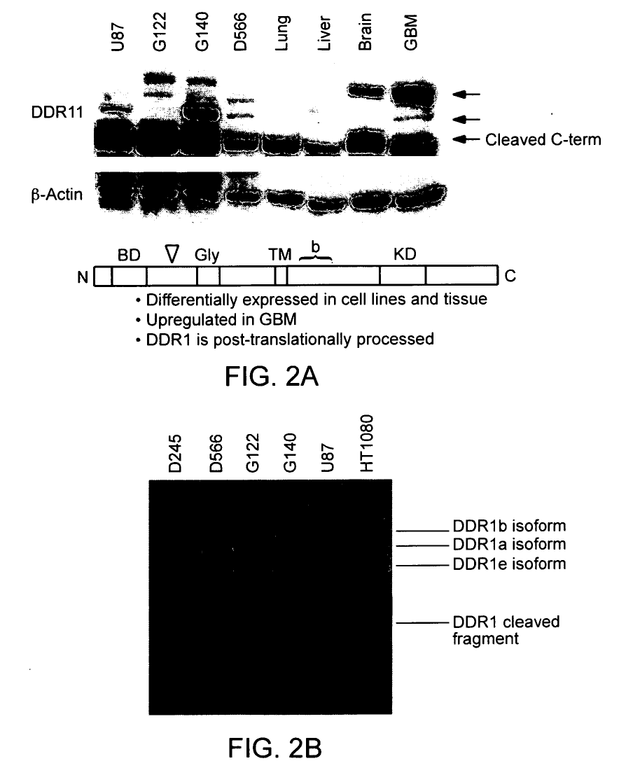

To document over expression of DDR1 protein in high grade Glioma, a collection of human Glioma derived cell lines, lung, liver, brain and GBM tissue was tested (FIGS. 2A and 2B). Western Blot analysis using an antibody to the C-terminal region of DDR1 detected 3 bands of approximately 125 kDa and 110 kDa, corressponding to DDR1a/1b and DDR1e isoforms and a 62 kd kDa transmembrane protein. Lysates of Glioma derived cells in culture and tissue samples from normal brain, lung, liver, and gliobastoma were immunoblotted, and probed with polyclonal anti-DDR1 Ab (C-20). Lysates were also analyzed by Western blot for β-actin as a control for protein loading. This analysis demonstrates that DDR1 is upregulated in GBM tumor tissue and is differentially expressed in glioma cell lines.

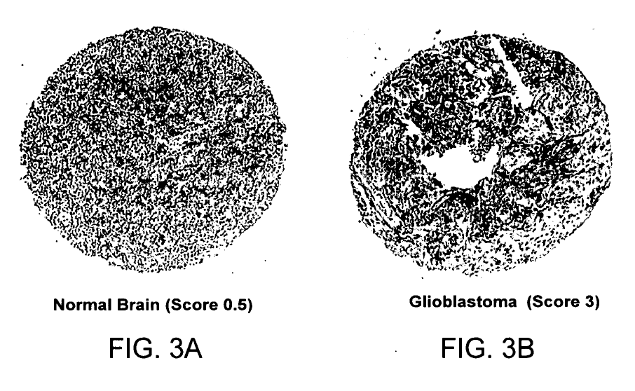

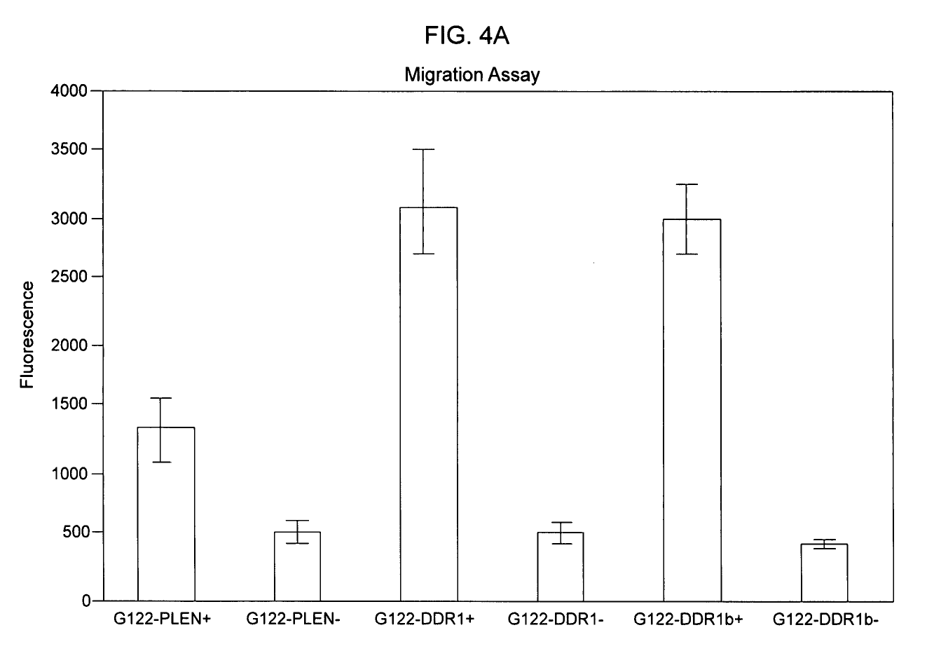

The localization of DDR1 protein was analyzed by immunohistochemistry on paraffin sections of primary tumors (FIGS. 3A and 3B and Table 1). In this study, 15 out of 19 high grade astrocytoma tumors (79%) stained positive for DDR1, and very low level of DDR1 expression was identified in normal brain sections (FIGS. 3A, 3B and Table 1). Consistent with Western blot analysis, these results demonstrate an upregulation of DDR1 protein in high grade Glioma tumor tissue. Therefore, expression of DDR1 by Glioma tumors demonstrates that DDR1 is a potentially useful diagnostic and therapeutiuc marker of tumor cells within the CNS.

The expression of DDR1 in primary brain tumors was tested for specificity of tumor type by staining with anti-DDR1 (C-20, C-terminal Antibody, Santa Cruz Biotechnology Inc.). The results are shown in Table 2. Astrocytomas grade III and grade II were strongly correlated with DDR1 expression, while other types of brain tumors had low or no expression of DDR1. DDR1 was also found to be overexpressed in other tumors, including lymphomas.

TABLE 2

| Tumor Type | Incidence |
|---|---|
| Astroytoma III | 3 of 3 (100% |
| Astrocytoma II | 3 of 4 (75%) |
| Astrocytoma IV | 2 of 2 (100%) |
| Meningioma IV | 0 of 2 (0%) |
| Meningioma I | 3 of 8 (37%) |
| Schwannoma I | 0 of 4 (0%) |
| Medulloblastoma | 0 of 2 (0%) |
| Glioblastoma Multiforme Garde III | 2 of 2 (100%) |
| Glioblastoma Multiforme Garde IV | 22 of 24 (90%) |

Overexpression of DDR1 in other Tumor Tissues

| Cancer Tissue | Histology | Positive Tumors | Normal Tissue |
|---|---|---|---|
| Breast | Adenocarcinoma | 4 of 16 (25%) | 1 of 7 (14%) |
| Ovary | Cystadenocarcinoma | 4 of 9 (44%) | 0 of 3 (0%) |
| Endometrium | Adenocarcinoma | 2 of 7 (28%) | ND |
| Gastric | Adenocarcinoma | 0 of 6 (0%) | 0 of 2 (0%) |
| Colon | Adenocarcinoma | 6 of 8 (75%) | 2 of 6 (33%) |
| Pancreas | Adenocarcinoma | 1 of 10* (10%) | 1 of 5 (20%) |
| Liver | Hepatocarcinoma | 0 of 5 (0%) | 1 of 1* (100%) |
| Renal/Pelvis | Transitional | 1 of 8 (12%) | 0 of 1 (0%) |
| | Carcinoma | | |
| Kidney | Renal Carcinoma | 3 of 14 (21%) | 4 of 5* (80%) |
| Bladder | Transitional | 6 of 17 (35%) | ND |
| | Carcinoma | | |
| Prostate | Adenocarcinoma | 6 of 13 (46%) | 1 of 7 (14%) |
| Skin | Melanoma | 3 of 5 (60%) | ND |
| Esophagous | Adenocarcinoma | 2 of 5 (40%) | ND |
| Lip/Tongue/ | Squamous | 18 of 28 (64%) | 1 of 7 (14%) |
| Mouth | | | |
| Paratoid | Mixed Tumor | 1 of 3 (33%) | 0 of 1 (0%) |
| Larynx | Squamous | 3 of 8 (37%) | 0 of 1 (0%) |
| Pharynx | Squamous | 1 of 3 (33%) | ND |
| Lymph Node | Lymphoma | 5 of 7 (71%) | 0 of 2 (0%) |
| Lung | Squamous/Adeno | 4 of 9 (44%) | 0 of 3 (0%) |

DDR1 promotes glioma cell migration through basement membrane. High grade Glioma tumors are notable for its highly migratory and invasive behavior. The primary cause of local recurrence and therapeutic failure in the treatment of high grade astrocytomas is the invasion of tumor cells into the surrounding normal brain. To migrate, these cells must degrade the subendothelial matrix, which is rich in collagen IV and collagen I, the principal substrates for MMPs (Metalloproteases). To study the importance of DDR1 in cell migration, astrocytoma cells expressing empty vector (mock), DDR1a or DDR1b isoforms were generated.

Generation of stable cell lines over-expressing DDR1 and DDR1b. G122 astroctyoma cells were stably transfected with DDR1a, DDR1b, or vector alone. The cells were analyzed by immunoblotting with anti-DDR1 antibody, and shown to have the appropriate phenotype. Other Glioma cell lines (G140, D566, D245, U87) were also transfected to overexpress DDR1 isoforms.

The cDNA for DDR1a and DDR1b was cloned into a mammalian expression vector (pcDNA) and stably transfected into the glioblastoma cell lines using the fugene transfection method (Roche) according to the manufacturer's protocol. At 3 days after transfection, medium containing 500 μg/ml Geneticin (G418; Gibco BRL) was applied to select the transfectants. More than 40 Geneticin-resistant colonies were obtained and selected; the remaining cells were pooled after colony selection. The selected colonies were grown and expanded for further experiments, and maintained in medium containing 100 μg/ml Geneticin. Pools and DDR1-expressing clones were used for further experiments.

In order to confirm the overexpression of DDR1, immuoblotting was used to show that DDR1 was overexpressed in pools and clones when compared with the control, which was transfected with vector only. This showed that cells expressing DDR1a and DDR1b had increased levels of DDR1. In order to demonstrate phenotypic differences in DDR1-overexpressing glioma cells, the morphology of the transfectants was observed. Overexpression of DDR1 induced multilayered and bipolar-shaped cells, which are characteristics of transformed epithelial cells. The expression of DDR1 may be required for alterations in cell morphology and migration, since a change in the interaction between the cell and the ECM due to DDR1 can be a stimulatory signal for the cells to transform and to have a migratory character. Thus DDR1 may be a necessary factor in order for the cells to migrate and to change morphology, and is necessary for filopodia formation and cell locomotion. It was also observed that the DDR1b-overexpressing clones grow more slowly than control cells.

To characterize the functional properties of the extracellular domains of DDR1, we have generated stable Glioma cell lines expressing DDR1ex. The mammalian constructs are expressed from a pcDNA3.1/myc-His (−) (Invitrogen Life Technologies) backbone that includes a C-terminal peptide, containing a polyhistidine metal-binding tag and the c-myc-epitope. The incorporation of myc-epitope and polyhistidine tag allows biochemical assays to assess the expression and purification of the extracellular domains. These cell lines serve as suitable tools to express and characterize the targets in human glioma derived cell lines, where they are useful for screening antibodies.

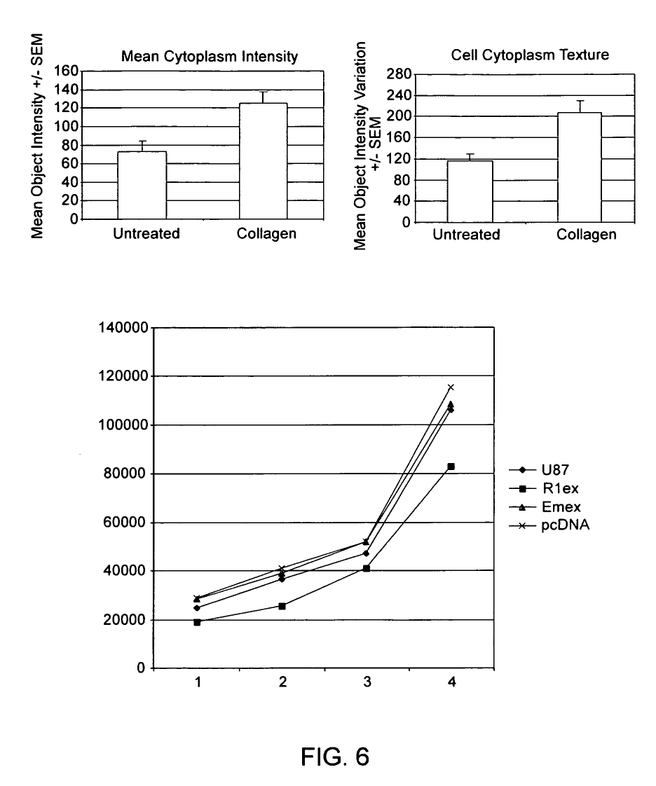

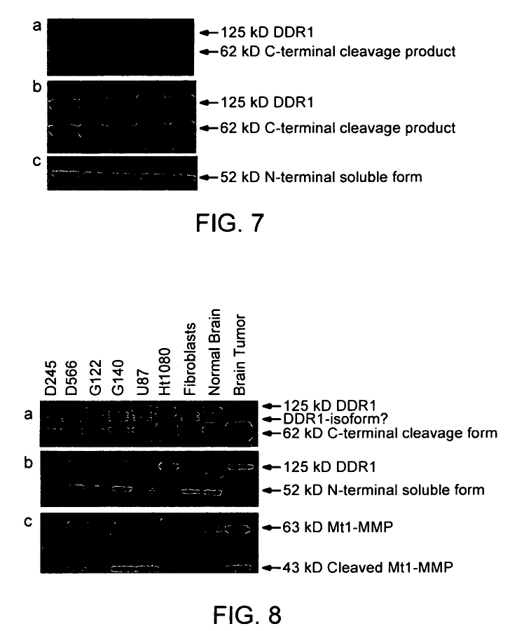

Overexpression of a DDR1 extracellular domain construct in glioma cells inhibited cell survival. U87 cells were plated onto a 96 well plate and growth of cells was measured using Cell Titer Glo Luminescent Cell Viability Assay (Promega). This assay is based on quantitaion of cellular ATP present, which signals the presence of metabolically active cells. The data is shown in FIG. 6.

To examine the role of DDR1 on cell proliferation, cell viability assays were performed in combination with RNAi transfection. Cell lysates from glioma cell lines after transient transfection with siRNA were tested for expression of DDR1, and found to have a knockdown of DDR1 expression.

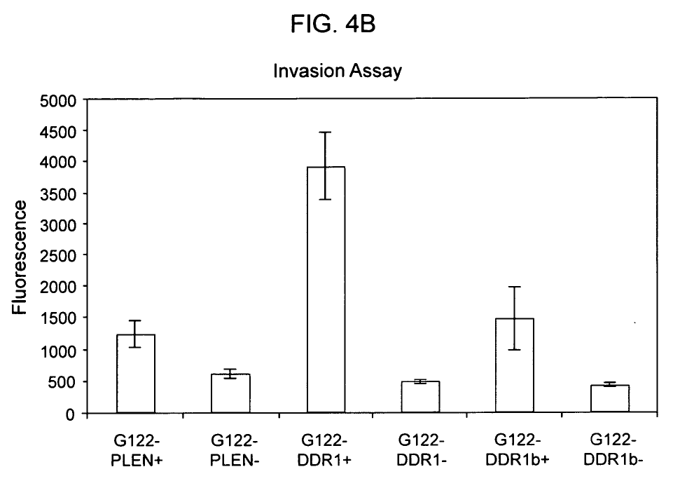

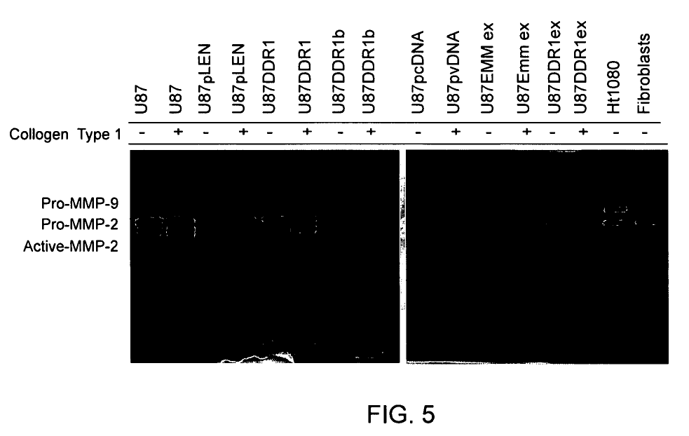

Migration assays. Overexpression of DDR1a isoform increased cell migration and invasion through Matrigel. Cells expressing DDR1a, DDR1b, vector alone (Mock) were suspended in DMEM plus 1% FBS and placed on top of FluoroBlok inserts (Becton Dickenson 8-μm pore size) noncoated or previously coated with Matrigel. DMEM with or without 5% serum was added to the lower chambers. After 4 hrs or 16 hours, migrating or invading cells adhering to the underside of the membrane were stained and fluorescence emitted by cells that have invaded through the matrigel was measured. The data is shown in FIG. 4. Similar studies were also performed with other cell lines overexpressing DDR1a and DDR1b, and a similar enhancement in invasiveness and migratory behaviour was seen. These cells also invaded through collagen 1, collagen IV and fibronectin matrices. Cells stably overexpressing DDR1a showed enhanced invasion through Matrigel compared to cells overexpressing DDR1b and emempty vector. These activities are directly related to increased expression of active matrix metalloproteinases.

Briefly, cells were trypsinized, and 100 μl of cell suspension (1×10⁶ cells/ml) were added in triplicate wells. Glioma cells expressing DDR1a, DDR1b, vector alone (Mock), DDR1ex, pcDNA (mock) were suspended in DMEM plus 1% FBS were placed on top of light opaque FluoroBlok inserts (Becton Dickenson) (8-μm pore size) previously coated with 100 μg/cm² of Matrigel (for invasion studies). DMEM containing plus 5% serum was added in the lower chambers. After 4 hrs or 16 hours, migrating cells adhering to the underside of the membrane were stained with 4 ug/ml calcein and florescence emitted by cells that have invaded through the matrigel was measured at λs of 530/590 nm using a CytoFlor plate reader. The number of Glioma cells expressing DDR1a displayed increased migration (FIG. 4A) compared to cells expressing vector alone or the isoform DDR1b. Similar results were seen when cells were plated on matrigel (FIG. (4B). Cells overexpressing DDR1a exhibited increased migration when compared to mock or cells expressing DDR1b. Ht1080 cells (a fibrosarcoma cell line) and human fibroblasts were used as positive and negative controls.

DDR1 overexpressing glioma cells reveal increased prescence of MMP-9, MMP-2 and MMP-1. Cells overexpressing DDR1a and DDR1b, DDR1 ex (extracellular domain construct) and Emmprin ex (extracellular domain construct), Ht1080 cells and human Fibroblasts, cells expressing empty vector were plated onto plates. After 24 hrs of plating, media was replaced with Serum-free medium with or without 20 ug/ml Type 1 Collagen and were incubated for 48 hrs at 37° C. Media from cells was collected and concentrated. 10 ug of media from each sample was resolved on a polyacrylamide gel (10%) containing 0.1% gelatin. Following electrophoresis, gels were washed twice with 5% Triton X-100 (30 min each). After washing, the gels were incubated for 24 h at 37° C. in the presence of 50 mM Tris-HCl, 5 mM CaCl₂, 5 μM ZnCl₂, pH 7.5, stained with Coomassie Brilliant Blue R-250 for 30 min and then destained. MMP-1, MMP-2 and MMP-9 production was induced by native type I collagen. Increased activation of pro-MMP-2 and Pro-MMP-1 was also seen with Type 1 Collagen.

Interestingly, human DDR1 displays the sequence RFRR (amino acids 304-307) in the stalk region, a sequence complying with the consensus site for furin endoproteases. However, studies with furin inhibitors suggest that this site is not involved in ligand-induced DDR1 shedding. As DDR1 cleavage is inhibited by batimastat, an enzyme of the family of MT-MMP is most likely involved in DDR1 shedding. Collagen binding to the discoidin domain of DDR1 may induce changes in the conformation of the stalk region, particularly in the sequence close to the plasma membrane. These conformational changes could open up a protease site. Ligand-induced tyrosine phosphorylation of DDR1 may induce clustering of a variety of signaling molecules, which could than recruit a protease molecule. Activation of DDR1 may also result in transcriptional up-regulation of proteases. The above studies with Glioma cells show an upregulation/activation of Mt1-MMP (Data not shown), MMP-9, MMP-2 and MMP-1. This up-regulation may include a protease that cleaves the receptor itself. Mt1-MMP is known to promote activation of pro-MMP-2 to its active form and enhance invasiveness in many tumor cells. Our Glioma cells lines expresses Mt1-MMP.

The ability of many tumor cells to invade their local environment and to metastasize from their primary site to vital organs such as liver, lung, and brain, is potentially life-threatening. Therefore, the critical event in tumor cell invasion is degradation of the extracellular matrix, because this process allows dissemination from the localized site. This matrix is composed of numerous structural macromolecules, including collagen types I, III, and IV. Most degradation is mediated by the matrix metalloproteinases (MMPs). Experimental and clinical studies suggest that elevated expression of MMPs correlates with tumor invasiveness and with an unfavorable prognosis. Considerable attention has focused on the role of the 72-kD gelatinase (MMP-2) and the 92-kD gelatinase (MMP-9), because of their ability to degrade type IV collagen in basement membrane. Production of these enzymes by numerous tumor cells has been documented and correlated with invasiveness. In addition to basement membranes, tumor cells must traverse the interstitial stroma, which is made up of collagens I and III. Thus, degradation of interstitial collagen is an essential component of the three-step process of invasion/metastasis: adhesion, degradation, and migration. Of significance is the fact that this degradation is accomplished most effectively by the interstitial collagenases, MMP-1, MMP-8, and MMP-13, and to some extent by MMP-2 and the membrane-type MMP, MT1-MMP (MMP14).

In summary, these findings demonstrate that expression of DDR1 in Glioma cells stimulates matrix degradation and basement membrane invasion. Using cell lines over expressing. DDR1a, and DDR1b, it is shown that cells overexpressing DDR1a show enhanced invasion through Matrigel, an activity that is related to increased expression of active matrix metalloproteinases.

Previous studies have described several types of host/tumor cell interactions that either mediate or augment tumor invasion by MMPs. These include secretion of MMPs by stromal cells in response to stimulation by tumor cells or, conversely, induction of MMP production by the tumor cells in response to host stimuli. Some of these mechanisms require direct contact between the stromal and tumor cells, whereas others do not. The present studies clearly indicate that a induction of MMP-1, MMP-2, MMP-9 and MT1-MMP by glioblastoma tumor cells facilitates tumor invasion through the type 1-collagen and Matrigel. Furthermore, invasion was inhibited by MMP inhibitor.

In a growing Glioma tumor, DDR1 may be important for the initial attachment of invasive cells to collagen. Following DDR1 activation, the cell/matrix contact is terminated by ectodomain cleavage, allowing further migration of the cell. Since the 62 kD transmembrane protein subunit of DDR1 is still tyrosine-phosphorylated following processing, the signalling pathways initially triggered by the full-length receptor remain active. The functional role of the DDR1 may depend on ligands other than collagen. Fibronectin can act to to phosphorylate DDR1 in glioma cells, and the 52 kD soluble protein or the DDR1 extracellular domain may function as a ligand by binding to DDR1.

DDR1 phosphorylation. Receptor tyrosine kinases (RTKs) play a key role in the communication of cells with their microenvironment. These molecules are involved in the regulation of cell growth, differentiation and metabolism. The protein encoded by DDR1 is a RTK that is widely expressed in normal and transformed epithelial cells and is activated by various types of collagen. This protein belongs to a subfamily of tyrosine kinase receptors with a homology region to the Dictyostelium discoideum protein discoidin I in their extracellular domain. Its autophosphorylation is stimulated by all collagens so far tested (type I to type VI). In response to collagen treatment, DDR1 is phosphorylated as a 125 kD (full length) protein, and a C-terminal cleavage product into a 52 kd soluble protein and a 62 kd tramsmembrane protein.

DDR1 activation in Glioma cells. DDR1 Glioma cells were stimulated with 10 ug/ml of Type1 collagen, 10 ug/ml Vitrogen, 10 ug/ml Fibronectin and 20 ng/ml EGF for 60 minutes. After stimulation, cells were lysed in RIPA buffer and resolved a 10% polyacrylamide gel (10%) gel. Lane 1, Nonstimulates, Lane 2, Type 1 Collagen (10 ug/ml, Lane 3 stimulated with Vitrogen (10 μg/ml), Lane 4 stimulated with human Fibronectin (10 μg/ml), Lane 5 stimulated with EGF 20 ng/ml) for 60 minutes. Cell lysates were analysed by anti-phosphotyrosine (4G10, Upstate Biotechnology) panel a, anti-DDR1 (C-20, Santa Cruz Biotechnology Inc.) panel b, and anti-DDR1 N-terminal H-126 (SCBT) panel c by western blotting. A tyrosine phosphorylated 62 kD and 125 kd protein is detected in panel A with anti-phosphotyrosine antibody, suggesting that stimulation with Type 1 Collagen, Fibronectin and EGF resulted in tyrosine phosphorylation of DDR1. An increase in DDR1 phosphorylation was seen with an increase in duration of collagen stimulation. An increase in 62 kD C-terminal fragment protein was seen with stimulation, panel b with C-terminal anti DDR1 antibody. DDR1 is proteolytically cleaved in response to ligand stimulation. A 52 kD soluble protein was detected in the media with H-126, a N-terminal anti-DDR1 antibody (panel c).

DDR1 internalization. Human Glioma derived were treated with soluble collagen I for 30 minutes and then stained for DDR1. The Cellomics ArrayScan fluorescent microscope instrument was used to identify and quantitate internalized DDR1 (FIG. 8). The data demonstrate that collagen I induces DDR1 to appear in the cytoplasm (mean cytoplasmic intensity increases) and the DDR1 specific fluorescent signal is punctuate (increased cytoplasmic texture), indicative of retention into endosomes. Measuring internalization of DDR1 demonstrates that a conjugated antibody can be specifically delivered to tumor cells and allow effective tumor cell killing.

### Example 2

### Discoidin Domain Receptor-1a (DDR1a) Promotes Glioma Cell Invasion and Adhesion in Association with Matrix Metalloproteinase-2

Invasion of glioma cells involves the attachment of invading tumor cells to extracellular matrix (ECM), disruption of ECM components, and subsequent cell penetration into adjacent brain structures. Discoidin domain receptor 1 (DDR1) tyrosine kinases constitute a novel family of receptors characterized by a unique structure in the ectodomain (discoidin-1 domain). These cell surface receptors bind to several collagens and facilitate cell adhesion. In the following example it is shown that DDR1 is overexpressed in glioma tissues using cDNA arrays, immunohistochemistry and Western blot analysis. Functional comparison of two splice variants of DDR1 (DDR1a and DDR1b) reveal novel differences in cell based glioma models. Overexpression of either DDR1a or DDR1b caused increased cell attachment. However, glioma cells overexpressing DDR1a display enhanced invasion and migration. We also detect increased levels of matrix metalloproteinase-2 in DDR1a overexpressing cells as measured by zymography. Inhibition of MMP activity using MMP inhibitors suppressed DDR1a stimulated cell-invasion. Similarly, an antibody against DDR1 reduced DDR1a mediated invasion as well as the enhanced adhesion of DDR1a and DDR1b overexpressing cells. These results demonstrate that DDR1a plays a critical role in inducing tumor cell adhesion and invasion, and this invasive phenotype is caused by activation of matrix metalloproteinase-2.

Materials and Methods

Glioma Cell Lines and Antibodies: Human U87-MG cells (ATCC) and G140 cells (kindly provided by Manfred Westphal, Frankfurt, Germany) were cultured in Dulbecco's modified Eagle's medium with 10% fetal bovine serum (Invitrogen), 100 U/mL penicillin/streptomycin. All cell lines were incubated at 37° C., 5% CO₂. DDR1 polyclonal antibody to the carboxy terminus: C-20, DDR1 monoclonal antibody to the extracelluar domain: 48B3 (19) (Santa Cruz Biotechnology, Inc.), monoclonal β-actin antibody (Abcam), monoclonal human integrin β1 antibody (Chemicon International, Inc.), phosphotyrosine antibody: 4G10 (Upstate Biotechnology, Inc.).

Functional Genomics: Library construction, hybridization and array analysis was performed as described previously. DDR1 accession numbers: Q08345; NM013994.

Immunohistochemistry: Land Mark Tissue MicroArray Low density Brain slides (Ambion) and IHC analysis was performed as per the manufacturer's instructions. All IHC reagents were purchased from Biogenex. The slides were analyzed and scored by US Labs, in a blinded outsourced study.

Immunoblotting: Cells lysates were prepared in RIPA buffer (0.1% SDS, 1% NP40, and 0.5% sodium deoxycholate in 1×PBS). Equal amounts of protein were resolved on 8% SDS-PAGE electrophoresis, blotted onto a nitrocellulose membrane (Bio-Rad), and incubated with a polyclonal anti-DDR1(C-20). The bands were visualized using the enhanced chemiluminescence (ECL) system (Amersham Pharmacia Biotech, Uppsala, Sweden). The blots were probed with β-actin antibody to confirm equal loading.

Stable expression of DDR1 into glioma cells: A cDNA for human DDR1a and DDR1b was overexpressed using a pLEN vector. The viral packaging cell line GP+e86 were transfected with the pLEN retroviral construct by the calcium phosphate method described elsewhere. After 2 days, media from transfected GP+e86 cells containing viral supernatant was filtered (0.45 μM) and added to G140 glioma cells in the presence of polybrene. After 24 h, G140 glioma cell were subjected to G418 selection and individual colonies were selected and screened for expression of DDR1a or DDR1b by Western blot analysis. Stably transfected G140 glioma cell were grown in Dulbecco's modified Eagles medium (DMEM) containing 10% fetal bovine serum, and 1% penicillin/streptomycin.

Immunoprecipitation for DDR1 phosphorylation: Rat tail collagen type 1 was purchased from Collaborative Biomedical Products (Bedford, Mass., USA). Cells were stimulated with 10 μg/ml soluble collagen type 1 for various periods of time. Cell lysates were prepared in RIPA buffer and immunoprecipitation was performed by incubating 1000 μg of cell extracts with DDR1 (C-20) antibody overnight at 4° C. Protein A beads (Santa Cruz Biotechnology: 50 μl) were added for 1 hr; the beads were washed three times with wash buffer and aliquots of the supernatant were subjected to SDS-PAGE. The blots were probed with monoclonal anti-phosphotyrosine antibody (4G10; Upstate Biotechnology Inc.) and visualized using the enhanced chemiluminescence (ECL) system (Amersham Pharmacia Biotech, Uppsala, Sweden). The blots were stripped and reprobed with a polyclonal antibody for DDR1 (C-20). Western Blot. The blots were probed with β-actin antibody to confirm equal loading.

Migration and Invasion Assays: Migration assays were performed using 24-well fluoroblok chambers (BD Biosciences, 8 μM pore size). Cells overexpressing DDR1a, DDR1 b, vector alone (Mock) and parental cells were trypsinized and 1×10⁶ cells were suspended in DMEM plus 1% serum. 100, 000 cells were placed in the upper chambers (Becton Dickenson-Fluoroblok plates-8 μm) in triplicate. DMEM with 5% serum was added to the lower chambers. After 4 hours, cells adhering to the underside of the membrane were stained with 4 μg/ml Calcein AM (Molecular Probes, Inc.) and fluorescence emitted by cells that have migrated through the chamber to the underside of the filter was measured at λs of 530/590 nm using a CytoFlor plate reader (Series 4000, Perspective Biosystems). For Invasion Assays: Rat tail Collagen type I (BD Biosciences, 10 μg/ml), human Collagen type IV (BD Biosciences, μg/ml), human Fibronectin (BD Biosciences, 5 μg/ml) were used to coat the underside of the chambers and Fluoroblok plates previously coated with Matrigel (BD Biosciences, 24 wells) were used. One-hundred μls of cells (1×10⁶ cells/ml) in DMEM plus 1% serum were placed in the upper chambers in triplicate. The cells were allowed to invade through the matrices at 37° C. for 16 hrs and stained with Calcein AM and fluorescence emitted by cells was measured at Xs of 530/590 nm using a CytoFlor plate reader (Series 4000, Perspective Biosystems).

Assays with MMP inhibitor: Glioma cells were grown for 24 hrs with GM6001, a broad range MMP-Inhibitor (Chemicon International, Inc.). Cells were trypsinized and (1×10⁶ cells/ml) incubated with MMP Inhibitor, GM6001 (25, 50 μM) for 1 hour at 37° C. with gentle agitation and 100,000 cells were placed into the top chamber (non-coated or previously coated with Matrigel or Collagen type 1) in triplicate. Migration or Invasion Assays were performed as described.

Adhesion Assays: Adhesion assays were performed by coating 96-well tissue culture plates with either Collagen type I (10 μg/ml) or 10 μg/ml BSA (Pierce). G140 cells were suspended in serum-free medium, 0.5% BSA, and plated at a density of 50,000 cells per well. Cells were allowed to attach for 0, 15, 30, 45, 60, 90 and 120 minutes. Non-adherent cells were rinsed off by washing the plates gently with PBS. Adherent cells were measured by adding Cell-titer Glo (Promega, Inc.) and then read on a luminometer at 570 nm. In some experiments, cells were incubated with mouse monoclonal anti-DDR1 antibody (48B3) or β1 integrin antibody for 1 hr at 37° C.

Zymography: Serum-free media from G140 cell lines were stimulated with Collagen type I (10 μg/mL and collected after 24 hr. In some experiments, GM6001 (25 μM, 50 μM) was added to the media. The media was mixed with Tris-Gycine SDS Sample buffer (2×) and 20 μg of protein was subjected to 10% SDS-polyacrylamide gel electrophoresis (Invitrogen, Inc.) using 0.1% gelatin. After electrophoresis, gels were incubated in 2.5% Triton X-100 (Renaturing Buffer, Invitrogen, Inc.) for 30 minutes twice to remove SDS, and replaced with 50 mM Tris-Hcl, pH 7.5 containing 0.15 M NaCl, 10 mM CaCl₂, and 0.02% NaN₃ (Developing Buffer, Invitrogen, Inc.) overnight and then stained with Coomassie Blue R-250 for 30 minutes.

Results

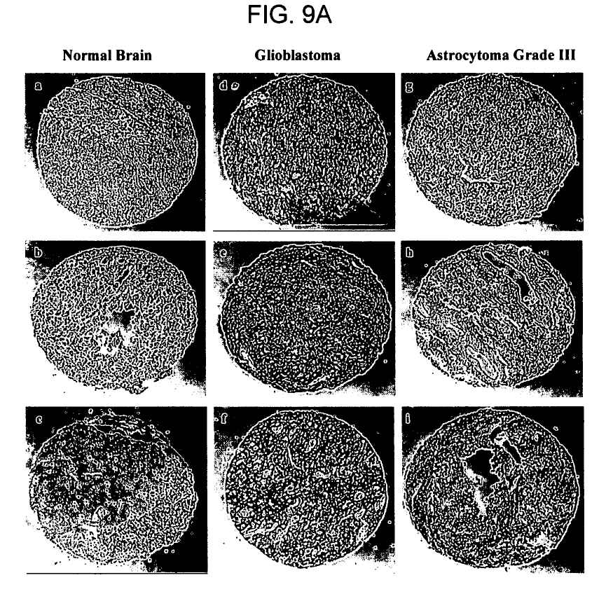

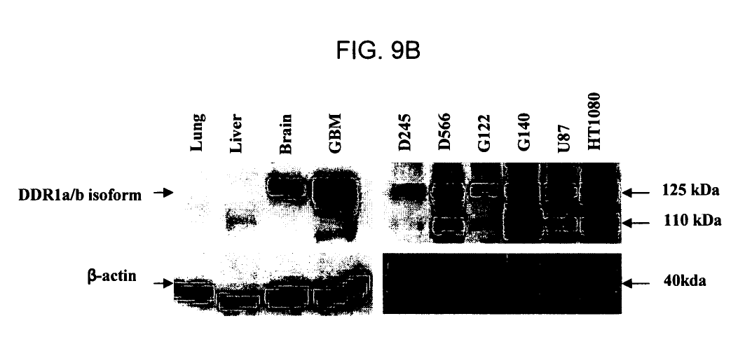

DDR1 is expressed in Glioma cells in culture and in vivo. Gene expression analysis of individual subtracted cDNA libraries generated from 14 GBM brain tumor samples and 6 normal brain samples identified DDR1 gene to be differentially upregulated by a factor of 1.8 fold with a p-value of 2.07E-07. The increase in mRNA expression level was also confirmed using quantitative PCR and in situ hybridization on a panel of normal brain and brain tumor samples (data not shown). To determine the expression level of DDR1 protein in gliobastoma, IHC analysis was performed using an anti-DDR1 antibody on a collection of human primary brain tumors and normal brain tissue (FIG. 9A). In this study 19 out of 24 glioblastoma samples (WHO Astrocytoma grade IV) (79%) stained positive for DDR1. Astrocytoma grade III (100%), and grade II (75%) tumor specimens also stained positive for DDR1. A low to moderate level of expression was identified in normal brain sections especially in Corpus Collosum and cerebellum. Consistent with our cDNA array data, these results clearly demonstrate upregulation of DDR1 protein in human astrocytic tumors.

The expression of DDR1 in lung, liver, brain and glioma tissue and various human glioma derived cell lines was tested by immunoblot (FIG. 9B). Using a polyclonal antibody to the C-terminal domain of DDR1, we detected a band at 125 kDa corresponding to DDR1a and DDR1b isoforms. A cleaved 62 kDa transmembrane proteins (beta-subunit) was also detected. A band around 110 kDa was also detected, which could correspond to the DDR1e isoform.

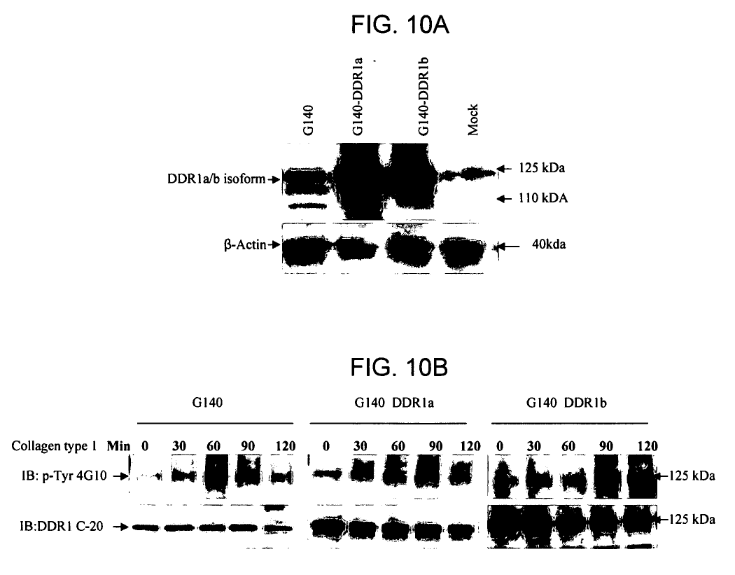

DDR1 is activated and tyrosine phosphorylated in response to Collagen type-1 in glioma cells. To test the role of DDR1 in glioma progression, a glioma cell model was developed by stably transfecting G140 cells with DDR1a and DDR1b cDNA in a retroviral vector. Independent clones were selected and grown. These cells were lysed, immunoblotted, and probed with anti-DDR1 C-terminal antibody. As shown in FIG. 10A, G140-DDR1a and G140-DDR1b cells overexpressed DDR1 (125 kDa) protein.

Collagen type 1 was added to G140 cells overexpressing DDR1a and DDR1b isoforms. We measured the levels of tyrosine phosphorylation of DDR1 receptor by immunoprecipitation with a DDR1 antibody, followed by an immunoblot analysis using the 4G10 phosphotyrosine antibody. As shown in FIG. 10B, autophosphorylation of endogenous DDR1 reached a peak at 60 min, and returned to a basal level by 120 min in G140 parental cells. Cells overexpressing DDR1a were also tyrosine phosphorylated with slow kinetics in response to soluble Collagen type 1 stimulation. Maximal DDR1a phosphorylation was reached after 60 minutes and remained high for up to 90 min in G140-DDR1a cells. In contrast, phosphorylation of G140-DDR1b peaked at 90 min and remained high up to 2 h. DDR1 phosphorylation in cells expressing the empty vector was similar to parental G140 cells. Some autophosphorylation of DDR1 in the absence of ligand was also seen, as indicated by the phosphotyrosine band visible at 0 min.

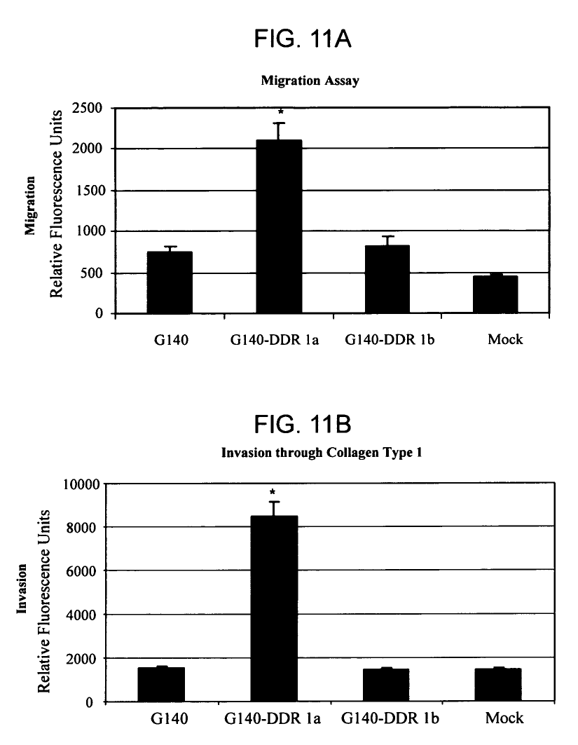

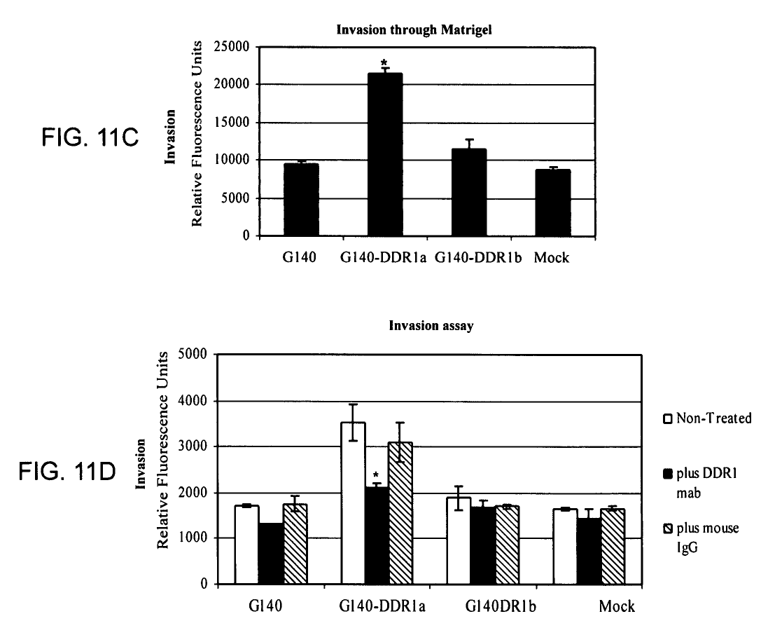

Overexpression of DDR1a stimulates Glioma cell Invasion in Vitro. The migration of DDR1a or DDR1 b overexpressing glioma cells was studied by a Transwell chamber migration assay. Overexpression of DDR1a resulted in a 2-3 fold increase in the number of migrating cells compared with parental G140 cells, G140-DDR1 b overexpressing cells and G140-mock cells (FIG. 11A). Next we performed, an invasion assay of G140 cells through membranes coated with collagen type 1 (FIG. 11B) or matrigel (FIG. 11C), G140-DDR1a overexpressing cells had a 3 to 4 fold increase in invasion compared with that of parental G140 cells. When an anti-DDR1 antibody (mab-48B3) (20 μg/ml) was included in the invasion assays through membrane coated with matrigel, the cell invasion stimulated by DDR1a overexpression was inhibited by more than 40% (FIG. 11D). Thus, DDR1a is capable of stimulating G140 cell invasion in vitro.

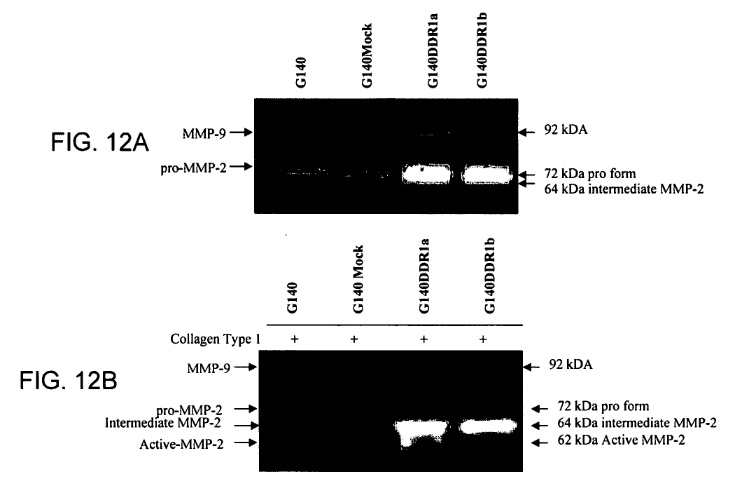

DDR1a stimulates activation of MMP-2 in human glioma cells. We next determined whether DDR1a expression and exposure to collagen type 1 could stimulate activation of MMP-2 in human glioma cells. To promote tumor invasion, pro-MMP-2, which is a secreted enzyme, has to be converted to fully activated MMP-2 through proteolysis. Conditioned media was collected from parental G140 and G140-DDR1a and G140-DDR1b overexpressing cells. As shown in FIG. 4A, an upregulation of the pro-MMP-9 (92 kDa), pro-MMP-2 (72 kDa) and of an intermediate form of MMP-2 (64 kDa) were found in the conditioned media of the G140-DDR1a and G140-DDR1b overexpressing cells compared to parental and mock cells. When these cells were stimulated with the collagen type 1 (10 μg/ml), an increased conversion of the intermediate (64 kDa) to the active form of MMP-2 (62 kDa) was found in G140-DDR1a overexpressing cells (FIG. 12B). By contrast, G140 parental cells, G140-DDR1b and G140 mock cells did not increase MMP-2 activation.

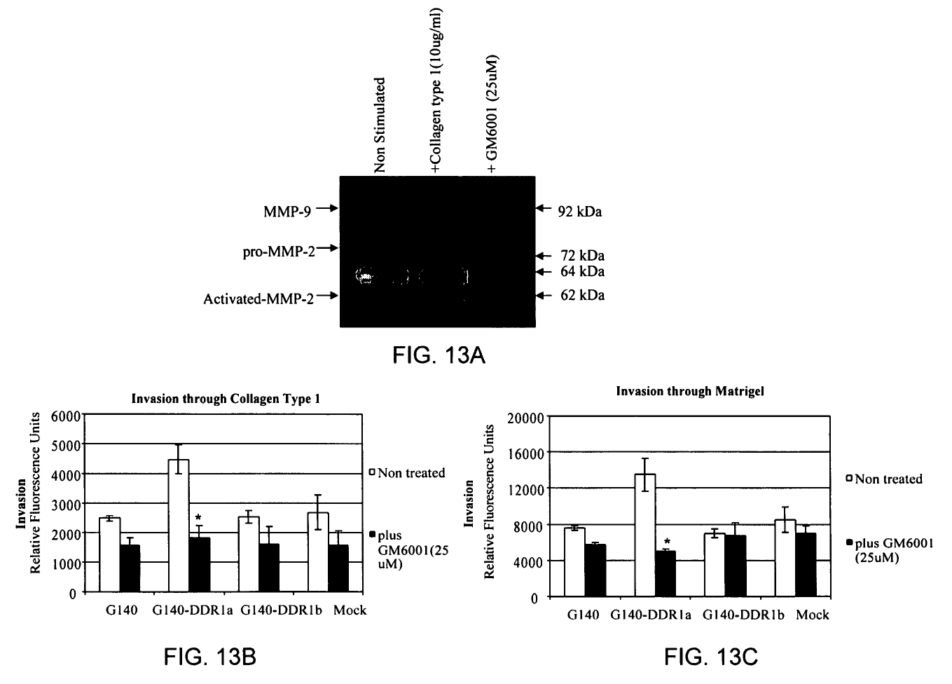

DDR1a Promotion of in Vitro Human Glioma Cell Invasion is Mediated Through Activation of MMP-2. We added GM6001, a broad range MMP inhibitor into cell cultures of G140 cells. Exposure to 25 μm of GM6001 effectively inhibited DDR1a stimulated enzymatic activities of MMP-2 in G140-DDR1a overexpressing cells (FIG. 13A). Next, we determined whether DDR1a-stimulated in vitro cell invasiveness was mediated by modulation of the activation of MMP-2 in glioma cells. We performed cell invasion assays by using G140-DDR1a and G140-DDR1b overexpressing and parental G140 cells, in the presence GM6001 (25 μM). GM6001 prevented DDR1a mediated invasion through collagen type 1 (FIG. 13B) and matrigel (FIG. 13C). Thus, DDR1a promotes human G140 cell invasion via activation of MMP-2 in glioma cells in vitro.

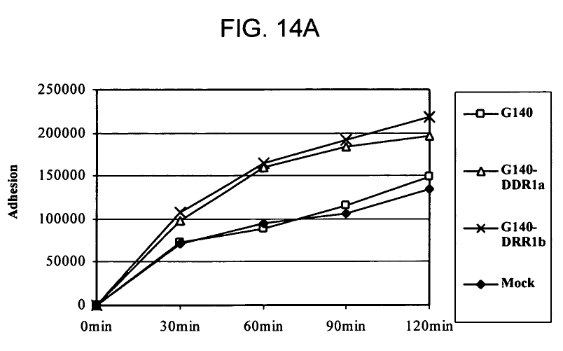

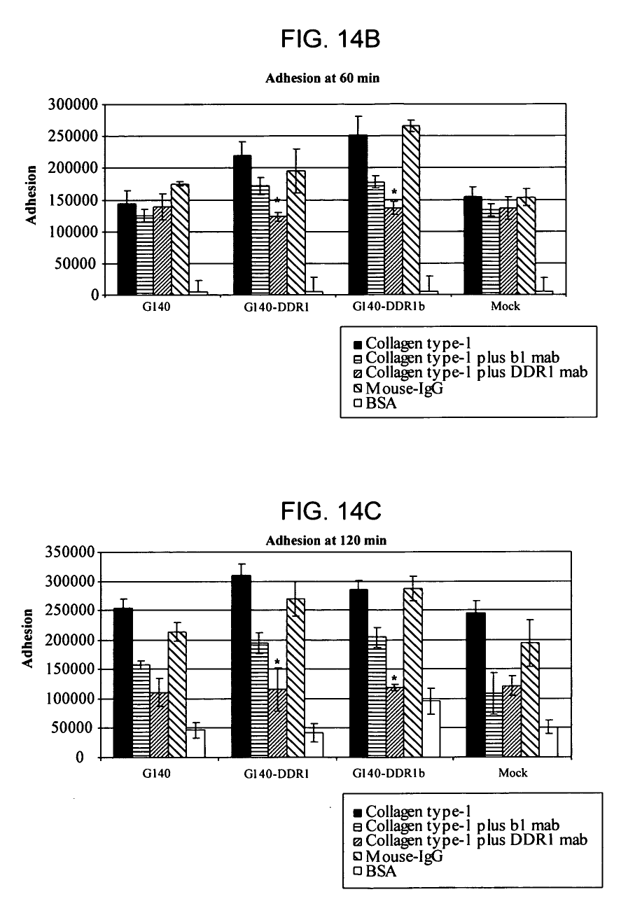

DDR1a and DDR1b-overexpressing cells showed enhanced attachment to type 1 collagen. To examine the role of DDR1 in glioma cell adhesion, G140 cells overexpressing DDR1a and DDR1b were used in attachment assays. Our experiments demonstrated a time-dependent increase in adhesion to collagen type I of G140 cell overexpressing DDR1a and DDR1b (FIG. 14A). In contrast, neither G140 parental cells, nor G140 cells expressing mock vector adhered well to collagen type 1. Similar results were seen with U87MG glioma cells. Attachment to fibronectin (5 μg/ml) was not significantly affected, demonstrating that attachment was specific for collagen type 1. Thus, our results have demonstrated significant enhancement in adhesion to collagen type 1 in glioma cells overexpressing DDR1a and DDR1b isoforms.

To test whether DDR1a and DDR1b overexpression in G140 cells influenced adhesion to collagen type 1, we blocked the DDR1 receptors on G140 cells using an antibody against human DDR1 (mab-48B3). None of the cell lines showed significant adherence to BSA-coated plates after 60 min of incubation. Small numbers of mock and parental G140 cells adhered to collagen type 1 coated plate at 60 min (FIG. 14B). In contrast, greater numbers of G140-DDR1a and G140-DDR1b overexpressing cells adhered to collagen type 1 coated plate. Increased adhesion of G140-DDR1a and G140-DDR1b cells was markedly impaired by a DDR1 antibody (FIG. 14B). A significant reduction in maximal cell adhesion attained was seen when compared to G140 parental cells or G140 cells mock cells. Integrins, especially α1β1 and α2β1 are known to play a role in the adhesion of cells to the collagens. Adherence of G140 cells overexpressing DDR1a and DDR1b was moderately inhibited by anti-integrin β1 antibody, thereby indicating that increased adhesiveness is mainly due to increased DDR1a and DDR1b expression. After, 120 min all four cell lines exhibited increased adherence to collagen type 1 (FIG. 14C). However, adherence of G140 parental cells or G140 cells expressing mock vector was inhibited by anti-integrin β1 blocking antibody, whereas the antibody had lesser effect on DDR1a and DDR1b overexpressing cells.

Discussion

The microenvironment of tumor cells is recognized to be pivotal to the growth of the tumor. Within this milieu, tumor cells interact with one another and with stromal cells via gap junctions, integrins, growth factors, and other mechanisms to enhance their survival, growth and invasiveness. The ability of glioma cells to invade diffusely throughout the CNS is a major factor that accounts for the high mortality and morbidity of the disease. The primary cause of local recurrence and therapeutic failure in the treatment of malignant gliomas is the invasion of tumor cells into the surrounding normal brain.

The current results provide evidence that glioma cells overexpress DDR1. We have addressed whether glioma cells overexpressing DDR1 can increase their invasiveness and adhesiveness. In this example, we present evidence that DDR1a but not DDR1b can directly induce glioma cell invasion. Overexpression of the DDR1a isoform by glioma cells conferred an aggressive invasive behavior in vitro where it increased their ability to invade through matrigel or Collagen type 1. Thus, DDR1a is capable of affecting glioma cell invasion.

Proteolytic ECM degradation is a key step in tumor invasion and metastasis. Although various proteinases are involved in the process, MMPs can degrade ECM components, and are believed to play a major role in invasion and metastasis. Among the MMPs, MMP-2 (gelatinase A) is considered to be especially important in the malignant behavior of the tumor cells. It has been reported that increased expression of MMP-2, MMP-9 and membrane type MT1-MMP is correlated with the invasive phenotype of glioma and other types of tumors. MT1-MMP is involved in the activation of MMP-2. Interestingly, we found that MT1-MMP was expressed in glioma cells, suggesting a role for it in DDR1-induced glioma invasion. Inhibition of MMP-2 expression suppresses the invasiveness of tumor cells in several model systems but molecules that induce MMP-2 activation during tumor development have not been well defined. Our results clearly demonstrate that DDR1a can function as such an inducer and thereby affect glioma cell invasiveness. Specifically, we have evaluated the activation of MMP-2 in glioma cells overexpressing the DDR1a isoform. We have observed that glioma cells, overexpressing DDR1a with resultant MMP-2 activation, displayed increased invasive behavior. Presence of GM6001, a broad range MMP-inhibitor prevented MMP activation, and also prevented the invasion and migration of glioma cells. The current results support the novel hypothesis that in glioma cells, DDR1a activation by collagen leads to conversion of pro-MMP-2 (72 kDa) into its active form (62 kDa). This facilitates the invasiveness of glioma cells by enhancing ECM degradation. Although MMPs are discussed here with respect to invasiveness, it should be emphasized that MMPs also regulate other aspects of tumorigenicity, such as survival, proliferation, and angiogenesis.

Glioma invasion involves cell adhesion and proteolytic degradation of the ECM. To the best of our knowledge, these studies are the first to demonstrate a functional role for the DDR1 in cell attachment of glioma cells. Consistent with these reports, G140 cells over expressing DDR1a and DDR1b exhibited a rapid adhesion to Collagen type 1, however parental cells and those expressing mock vector were weakly adherent. Furthermore, inhibition of DDR1 function using neutralizing antibody attenuated the adhesion of G140 cells overexpressing both DDR1a and DDR1b isoforms. These studies show that DDR1 is expressed in glioma cells and that DDR1 mediates selective adhesion to Collagen type 1 independent of β1-integrin.

Differences between the function of DDR1a and DDR1b in glioma cells suggest divergent signaling events. The slow kinetics of tyrosine phosphorylation of DDR1 in response to collagen stimulation has suggested a low-affinity interaction between collagen and DDR receptors. Previous studies have shown that activation of DDR1b, but not of DDR1a, causes recruitment of Shc and activation of MAPK. The differences in the timing and nature of DDR1a and DDR1b activation suggest that these isoforms may have different roles in glioma cells. Therefore, our work suggests that difference in the kinetics of receptor autophosphorylation observed between DDR1a and DDR1b is responsible for the different roles that the isoforms play in glioma cell invasion and adhesion. DDR1a and DDR1b, are overexpressed in a number of malignant tumor cells. In normal tissues, differential expression of DDR1 isoforms has been reported. In the present study, we show that glioma cells overexpressing DDR1a have a distinct function from DDR1b overexpressing cells in cell migration and invasion. However, both DDR1a and DDR1b facilitate adhesion of glioma cells to collagen type 1.

Patients with high-grade glioma have a very poor prognosis, as only 10% will survive beyond 2 years after initial diagnosis. A major cause of the high mortality is the inability to excise all tumor cells surgically, leading to the regrowth and dissemination of cells to form several distinct tumor masses within the CNS. Thus new therapies that suppress glioma invasiveness should improve the prognosis of patients with malignant gliomas. In summary, our findings provide a model in which expression of DDR1 in glioma cells stimulates extracellular matrix invasion and adhesion. The current results suggest that inhibition of DDR1a, resulting in the inhibition of MMP-2 activity, will improve treatment of glioma.

The foregoing is intended to be illustrative of the embodiments of the present invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein. All publications and patent applications are herein incorporated by reference to the same extent as if each individual publication or patent application was specifically and individually indicated to be incorporated by reference.

What is claimed is:

1. A method for the diagnosis or staging of a brain tumor, the method comprising:
determining the upregulation of expression of DDR1 mRNA or polypeptide in said brain tumor.

2. The method according to claim 1, wherein said astrocytoma is a grade II, grade III astrocytoma and grade IV astrocytoma.

3. The method according to claim 1, wherein said DDR1 is selected from the group consisting of the DDR1a isotype, DDR1b isotype, DDR1e isotype, soluble DDR1, and glioma specific isoforms.

4. A method to treat a brain tumor, the method comprising:
administering a therapeutic amount of a compound that binds to, or inhibits, DDR1.

5. The method according to claim 4, wherein said compound inhibits invasion, ligand binding, angiogenesis, survival, MMP production, ectodomain cleavage, biologic activity, and cell adhesion of astrocytoma cells.

6. The method of claim 4 wherein said compound is administered by intrathecal administration.

7. The method of claim 6, wherein said compound is formulated for retention and stability in the brain.

8. The method of claim 6 wherein said compound is administered by intravascular administration.

9. The method of claim 6, wherein said compound is a specific binding partner for DDR1.

10. The method according to claim 9, wherein said specific binding partner is conjugated to a cytotoxic moiety.

11. The method according to claim 9, wherein said binding partner is internalized by said astrocytoma cell.

12. The method according to claim 9, wherein said specific binding partner is an antibody.

13. The method according to claim 12, wherein said antibody binds to an epitope selected from the group consisting of the discoidin domain; the F5/8 type C domain; the RFRR protease recognition site; amino acid sequence 380-416, and the gly-pro rich domains.

14. The method according to claim 9, wherein said specific binding member is a collagen fragment that binds to DDR1.

15. The method according to claim 9, wherein said specific binding member is a soluble fragment of DDR1 that forms a homotypic dimmer with membrane bound DDR1.

16. The method according to claim 9, wherein said specific binding member is a fibronectin fragment that binds to DDR1.

17. A method of screening candidate agents for effectiveness against an astrocytoma, the method comprising:
combining a candidate biologically active agent with a DDR1 polypeptide; or a cell comprising a nucleic acid encoding and expressing a DDR1 polypeptide; and
determining the effect of said agent on DDR1 activity, wherein agents that modulate polypeptide activity provide for molecular and cellular changes in brain tumor cells.

18. The method according to claim 17, wherein said biologically active agent modulates activity of said polypeptide.

19. The method according to claim 17, wherein said activity is DDR1 mediated modulation of matrix metalloprotease activity.

20. The method according to claim 17, wherein said activity is invasion of extracellular matrix.


SEQUENCE LISTING (6 sequences)

SEQ ID NO: 1 - DNA, 3838 bp, Homo sapiens
  CDS (337)..(2964)
ggcttaggaagtattaactgatctctgccctagttctcatgtgttaaatatggatagtaatagtatctaccttatgaagtgactgtgaagataaaattatggattctgtttaagggtttaggccagtgtctggcacaggggaagcattctaaaaatatagctgatgctgttaaacaatgactgttgttgttgttttactgttattatccccaaagcggcccattctgtctgttgctgtcagctatgactcagtcccctgattaacttacgcaccacccattttatcccctgcagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcgctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggcagctcaccgacgagcaggtcatcgagaacgcgggggagttcttccgggaccagggccggcaggtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaa

SEQ ID NO: 2 - protein, 875 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSAERRVLEEELTVHLSVPGDTILINNRPGPREPPPYQEPRPRGNPPHSAPCVPNGSAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 3 - DNA, 3952 bp, Homo sapiens
  CDS (337)..(3078)
ggcttaggaagtattaactgatctctgccctagttctcatgtgttaaatatggatagtaatagtatctaccttatgaagtgactgtgaagataaaattatggattctgtttaagggtttaggccagtgtctggcacaggggaagcattctaaaaatatagctgatgctgttaaacaatgactgttgttgttgttttactgttattatccccaaagcggcccattctgtctgttgctgtcagctatgactcagtcccctgattaacttacgcaccacccattttatcccctgcagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcgttgctgctctccaatccagcctaccgcctccttctggccacttacgcccgtccccctcgaggcccgggcccccccacacccgcctgggccaaacccaccaacacccaggcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggtcagctcaccgacgagcaggtcatcgagaacgcgggggagttcttccgggaccagggccggcagtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaa

SEQ ID NO: 4 - protein, 913 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGSAHRRAGHRERGGVLPGPGPAVYLSRPPACPQGLYELMLRCWSRESEQRPPFSQLHRFLAEDALNTV

SEQ ID NO: 5 - DNA, 3970 bp, Homo sapiens
  CDS (337)..(3096)
ggcttaggaagtattaactgatctctgccctagttctcatgtgttaaatatggatagtaatagtatctaccttatgaagtgactgtgaagataaaattatggattctgtttaagggtttaggccagtgtctggcacaggggaagcattctaaaaatatagctgatgctgttaaacaatgactgttgttgttgttttactgttattatccccaaagcggcccattctgtctgttgctgtcagctatgactcagtcccctgattaacttacgcaccacccattttatcccctgcagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcgttgctgctctccaatccagcctaccgcctccttctggccacttacgcccgtccccctcgaggcccgggcccccccacacccgcctgggccaaacccaccaacacccaggcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccagcttctccttgttctccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggtcagctcaccgacgagcaggtcatcgagaacgcgggggagttcttccgggaccagggccggcagtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaa

SEQ ID NO: 6 - protein, 919 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNASFSLFSRNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGSAHRRAGHRERGGVLPGPGPAVYLSRPPACPQGLYELMLRCWSRESEQRPPFSQLHRFLAEDALNTV